(19) United States
(12) Patent Application Publication
(10) Pub. No.: US 2011/0207117 A1
(43) Pub. Date: Aug. 25, 2011

(54) GENERATION OF PRODUCTION STRAINS THAT EFFICIENTLY EXPRESS NUCLEAR TRANSGENES

(76) Inventors: Ralph Bock, Schwielowsee (DE); Daniel Karcher, Postdam-Golm (DE); Juliane Neupert, Postdam (DE)

(21) Appl. No.: 12/994,133

(22) PCT Filed: May 25, 2009
(86) PCT No.: PCT/EP2009/003684

§ 371 Date: Apr. 11, 2011

(30) Foreign Application Priority Data
May 23, 2008 (EP) 08009487.3

(51) Int. Cl. C12Q 1/68; C12N 15/87; C12P 21/00; C12P 19/34; C12N 5/10; C12N 1/00

(52) U.S. Cl. 435/6.1; 435/471; 435/468; 435/455; 435/446; 435/441; 435/69.1; 435/91.3; 435/325; 435/419

(57) ABSTRACT

The present invention relates to a method of generating eukaryotic cells suitable for the expression of transgenes in said cells comprising (a) introducing a nucleic acid encoding a selectable marker responsive to a selecting agent into the nucleus of cells, wherein the level of expression of said selectable marker is proportional to the level of phenotypic responsiveness to said selecting agent; (b) selecting, among the cells obtained in step (a), for cells with a detectable expression of said selectable marker; (c) optionally propagating the cells selected for in step (b); (d) mutagenizing the cells selected for in step (b) or propagated in step (c) or allowing for the appearance of spontaneous mutations in the cells selected for in step (b) or propagated in step (c); and (e) selecting for cells displaying an increased expression of said selectable marker compared to the expression obtained in step (b). The present invention furthermore relates to a eukaryotic cell produced by the method of the present invention, a method of producing a compound of interest in a cell produced with the method of the present invention comprising (a) introducing a nucleic acid encoding (i) the compound of interest which is a protein or an RNA; or (ii) a protein necessary to synthesize said compound of interest; and optionally a selectable marker responsive to a selecting agent into said cell; (b) expressing said protein in the cell; and (c) isolating the compound of interest produced; and a kit comprising (a) a cell obtainable by the method of the invention and optionally a vector optimized for protein expression in said cell; or (b) the cell of the invention.

### RELATED PATENT APPLICATIONS

This patent application is a national stage of international patent application number PCT/EP2009/003684, filed on May 25, 2009, which claims the benefit of European patent application no. 08009487.3, filed on May 23, 2008, entitled “GENERATION OF PRODUCTION STRAINS THAT EFFICIENTLY EXPRESS NUCLEAR TRANSGENES.” The entire content of each of these patent applications hereby is incorporated by reference herein, including all text, drawings and tables.

### SEQUENCE LISTING

The instant application contains a Sequence Listing which has been submitted in ASCII format via EFS-Web and is hereby incorporated by reference in its entirety. Said ASCII copy, created on Nov. 15, 2010, is named VOS-1002-US.txt and is 3,093 bytes in size.

The present invention relates to a method of generating eukaryotic cells suitable for the expression of transgenes in said cells comprising (a) introducing a nucleic acid encoding a selectable marker responsive to a selecting agent into the nucleus of cells, wherein the level of expression of said selectable marker is proportional to the level of phenotypic responsiveness to said selecting agent; (b) selecting, among the cells obtained in step (a), for cells with a detectable expression of said selectable marker; (c) optionally propagating the cells selected for in step (b); (d) mutagenizing the cells selected for in step (b) or propagated in step (c) or allowing for the appearance of spontaneous mutations in the cells selected for in step (b) or propagated in step (c); and (e) selecting for cells displaying an increased expression of said selectable marker compared to the expression obtained in step (b). The present invention furthermore relates to a eukaryotic cell produced by the method of the present invention, a method of producing a compound of interest in a cell produced with the method of the present invention comprising (a) introducing a nucleic acid encoding (i) the compound of interest which is a protein or an RNA; or (ii) a protein necessary to synthesize said compound of interest; and optionally a selectable marker responsive to a selecting agent into said cell; (b) expressing said protein in the cell; and (c) isolating the compound of interest produced; and a kit comprising (a) a cell obtainable by the method of the invention and optionally a vector optimized for protein expression in said cell; or (b) the cell of the invention.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Expression of recombinant proteins or nucleic acids in cells not naturally harbouring said proteins or nucleic acids has become a valuable tool in research as well as for large-scale production of various compounds such as pharmaceuticals or enzymes. Recombinant expression in prokaryotes has by now been optimized and is useful if the compounds produced are not naturally subjected to chemical modifications after transcription and/or translation. Compounds expressed in prokaryotes and not modified in that way do very often not exert their biological activity.

On the other hand, recombinant expression in eukaryotic cells has the major drawback that expression yields are usually low or that expression cannot even be achieved at all. This is due to a number of mechanisms such a gene silencing used by these cells to suppress gene expression. In view of the potential use of eukaryotic cells with all modifications effected to expression products in these cells, they have become a target for the production of “green chemicals”, biofuels and recombinant proteins, such as biopharmaceuticals (vaccines, antibodies). This applies to promising eukaryotic organisms such as various plants, e.g. algae or plant cell cultures, fungi or mammalian cells.

Being single-celled algae that contain a single large chloroplast, algae of the genus Chlamydomonas represent one of the simplest photosynthetic eukaryotes. They can reproduce sexually or asexually and can grow photoautotrophically, heterotrophically or mixotrophically. Among Chlamydomonas species, the green alga Chlamydomonas reinhardtii has become a superb model organism for a wide range of biological questions, including, for example, flagella function, photobiology and photosynthesis research (Hippler et al., 1998; Harris, 2001; Pedersen et al., 2006; Schmidt et al., 2006). Moreover, Chlamydomonas reinhardtii combines a powerful genetics with the availability of unique genetic and genomic resources: All three genomes are fully sequenced (nuclear, plastid and mitochondrial; Merchant et al., 2007), large mutant collections have been established and all three genomes are amenable to genetic manipulation by transformation (Hippler et al., 1998; Remade et al., 2006).

The recent completion of the Chlamydomonas reinhardtii genome sequencing project (Merchant et al., 2007) has provided novel insights into the evolution of photosynthetic eukaryotes and paved the way to exploit the alga as a model system in plant post-genomics research. Most tools required for systematic functional genomics are available in Chlamydomonas reinhardtii, including high-frequency transformation protocols (Kindle, 1990), efficient methods for chemical and insertional mutageneses (Dent et al., 2005) and workable protocols for RNA interference (RNAi; Rohr et al., 2004). However, as observed for many other eukaryotic cells, a major obstacle to Chlamydomonas reinhardtii research is posed by the disappointingly poor expression of transgenes from the alga's nuclear genome.

In view of the above, there exists a need for strains of eukaryotic cells or eukaryotic organisms with an increased expression of transgenes.

Accordingly, the present invention relates to a method of generating eukaryotic cells suitable for the expression, preferably increased expression of transgenes in said cells comprising (a) introducing a nucleic acid encoding a selectable marker responsive to a selecting agent into the nucleus of cells, wherein the level of expression of said selectable marker is proportional to the level of phenotypic responsiveness to said selecting agent; (b) selecting, among the cells obtained in step (a), for cells with a detectable expression of said selectable marker; (c) optionally propagating the cells selected for in step (b); (d) mutagenizing the cells selected for in step (b) or propagated in step (c) or allowing for the appearance of spontaneous mutations in the cells selected for in step (b) or propagated in step (c); and (e) selecting for cells displaying an increased expression of said selectable marker compared to the expression obtained in step (b).

Expression of genes contained in the nucleus takes place in two steps: Transcription is effected in the nucleus and results in an mRNA product exported into the cytosol where it is translated into amino acid sequences forming peptides or proteins and possibly is post-translationally modified. In the context of the present invention, the term “expression” refers to the process resulting in an expression product which can be a protein or peptide, also referred to as “(poly)peptide”, or a nucleic acid which is not translated into a (poly)peptide such as certain RNA species, e.g. rRNA, siRNA, shRNA, miRNA, ribozymes, riboswitches or antisense RNA. “Expression of transgenes” is the production of the expression product of a foreign gene introduced into a cell in said cell. The term “increased expression” as used in the context of the present invention refers to a higher amount of the expression product of a transgene produced in eukaryotic cells in step (d) as compared to production levels of said transgene which could be observed in said cells after they were subjected to step (b). For example, if low expression of a specific transgene was detectable in cells after step (b), “increased expression” preferably denotes an increase in the amount of expression product obtained of at least 10%, preferably at least 25%, more preferred at least 50%, even more preferred at least 100%, such as at least 200%, at least 300%, at least 400% and most preferably at least 500%. If the transgene is a gene heterologous to the cell, i. e. originating from a different strain or species, an increase is generally meant to be an improved expression over expression yields obtained with prior art methods, i. e. cells or organisms not subjected to the methods of the present invention. Alternatively, an increase is an amount of expression product detectable over background level using conventional detection methods. Preferably, an increase amounts to at least 0.0001% such as at least 0.001% or at least 0.01%, more preferred at least 0.05%, even more preferred at least 0.1% such as at least 0.2% of total protein expressed in the respective cell. An increase can be measured by comparing yields of prior art methods with yields obtained with the method of the present invention. The measurement can be effected using methods well known in the art and/or described throughout this specification. The percentage of increase generally corresponds to the values mentioned above.

Generally, the gene encoding the protein or nucleic acid to be expressed is introduced into the cell in the form of an expression vector. Conditions for expressing proteins in different organisms or cells of different species are well known in the art and depend on the protein expressed as well as the cell used.

A typical eukaryotic expression vector contains a promoter element, which mediates the initiation of transcription of mRNA, the protein coding sequence, and signals required for the termination of transcription and polyadenylation of the transcript. Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Possible examples for regulatory elements ensuring the initiation of transcription comprise the cytomegalovirus (CMV) promoter, RSV-promoter (Rous sarcoma virus), the lacZ promoter, the gal10 promoter, human elongation factor 1a-promoter, CMV enhancer, CaM-kinase promoter, the Autographa californica multiple nuclear polyhedrosis virus (AcMNPV) polyhedral promoter or the SV40-enhancer. Examples for plant promoters are a constitutive promoter such as the fig wart mosaic virus 35S promoter, the cauliflower mosaic virus promoter, CaMV35S promoter, or the MAS promoter, or a tissue-specific promoter, such as the carnation petal GST1 promoter or the Arabidopsis SAG12 promoter (See, for example, J. C. Palaqui etal., Plant Physiol., 112: 1447-1456 (1996); Morton et al., Molecular Breeding 1: 123-132 (1995); Fobert et al., Plant Journal, 6: 567-577 (1994); and Ganetal, Plant Physiol., 113: 313 (1997)). Yeast promoters are e.g. the Gal10 promoter or the TPI triosephosphat isomerase promoter. Examples for transcription termination signals are the CaMV35S or Nos terminators, the SV40-poly-A site or the tk-poly-A site or the SV40, lacZ and AcMNPV polyhedral polyadenylation signals, downstream of the polynucleotide. Moreover, elements such as origin of replication, drug resistance genes, regulators (as part of an inducible promoter) or internal ribosomal entry sites (IRES) may also be included.

The term “(poly)peptide” as used herein describes a group of molecules which comprises the group of peptides, consisting of up to 30 amino acids, as well as the group of polypeptides also termed proteins, consisting of more than 30 amino acids. (Poly)peptides may further form dimers, trimers and higher oligomers, i.e. consisting of more than one (poly)peptide molecule. (Poly)peptide molecules forming such dimers, trimers etc. may be identical or non-identical. The corresponding higher order structures are, consequently, termed homo- or heterodimers, homo- or heterotrimers etc. The terms “(poly)peptide” and “protein” also refer to naturally modified (poly)peptides/proteins wherein the modification is effected e.g. by glycosylation, acetylation, phosphorylation and the like. Such modifications are well known in the art.

A “nucleic acid”, in accordance with the present invention, includes DNA, such as cDNA or genomic DNA, and RNA, both sense and anti-sense strands as well as conventionally modified or derivatized nucleic acid molecules.

In this regard, a nucleic acid being an expression product is preferably an RNA, whereas a nucleic acid to be introduced into a cell is preferably DNA.

A “transgene” is a nucleic acid molecule that has been transferred from one organism to another. The term “transgene” preferably describes a segment of DNA that has been isolated from one organism or produced semi-synthetically or synthetically according to a nucleic acid sequence found in said organism and which is introduced into a different organism. This non-native segment of DNA, the “transgene” in the new host organism, may retain the ability to be expressed as RNA or peptide or protein in the transgenic organism. A “transgene” according to the above definition can be a cDNA or a gene as naturally present in genomic DNA including non-coding regions such as introns. Alternatively, a transgene can be a DNA sequence encoding an RNA species. In this case, a transgene has a minimal length of at least 25 nucleotides, preferably at least 40 nucleotides, more preferably at least 60 nucleotides. If the transgene gives rise to an siRNA, shRNA or miRNA, the transcription product is preferably in the range of 17 to 27, more preferred 19 to 21 nucleotides long, forms a double strand and optionally has one or two overhangs, as is known in the art. In context of the present invention, a transgene may be a gene originating from a different species than that the transgenic organism belongs to but may also be a gene isolated from one individual (cell) of a species and introduced into a different individual (cell) of the same species. The latter includes e.g. mutant alleles of a gene found in certain individuals of a species. In other words, transgenes introduced into cells in accordance with the present invention can be homologous or heterologous nucleic acid sequences.

The term “introducing a nucleic acid” refers to the application of a nucleic acid to cells and its subsequent uptake and incorporation into the genetic information of said cells, in particular in the nucleus. Depending on the species of the cell, i.e. what type of eukaryote is concerned, i.e. to which class of organisms it belongs (plants, fungi, animals, e.g. vertebrates or, more specifically, mammals), different terminologies are used to denote this process. In general, the genetic alteration of a cell resulting from the introduction/uptake and expression of foreign genetic material is termed “transformation” but the term is also used to describe only non-viral DNA transfer in non-animal eukaryotic cells such as fungi, algae and plants. The term “transduction” is used for genetic alterations resulting from introduction of DNA by viruses. “Transformation” of animal cells, in particular mammalian cells, is usually called “transfection”. Yeasts and fungi may be transformed by commonly known methods, such as the lithium acetate/single-stranded carrier DNA/polyethylene glycol methods (Gietz and Woods, 2002). Another alternative for the transformation of yeasts is the Frozen Yeast Protocol resulting in frozen yeast cells that are competent for transformation after thawing (Schiestl et al., 1993). Gene gun transformation is carried out with gold or tungsten nanoparticles coated with DNA which are shot into e.g. fungal cells, plant cells or plant embryos, thereby transforming them. The transformation efficiency with this method is lower in plants than for bacterially mediated transformation, but most plants can be transformed with this method. By protoplast transformation, fungal spores or plant cells can be converted to protoplasts by removing their cell wall, and can then be soaked in a solution containing DNA and transformed. Transformation of plant cells devoid of a cell wall can also be carried out using the glass-beads-method (Kindle et al, 1990).

Agrobacterium mediated transformation is the easiest and most simple plant transformation (An, 1987). For example plant tissue (often leaves) are cut in small pieces, e.g. 10×10 mm, and soaked for 10 minutes in a fluid containing suspended Agrobacterium. Some cells along the cut will be transformed by the bacterium, that inserts its DNA into the cell. Placed on selectable rooting and shooting media, the plants will regrow. Some plant species can be transformed just by dipping the flowers into suspension of Agrobacterium and then planting the seeds in a selective medium. Agrobacterium can also be transformed using electroporation (Weigel and Glazebrook, 2006).

In viral transduction of plant cells, the desired genetic material is packaged into a suitable plant virus and the resulting virus is allowed to infect the plant. If the genetic material is DNA, it can recombine with the chromosomes to produce transformant cells. Transfection of animal cells typically involves opening transient pores or ‘holes’ in the cell plasma membrane, to allow the uptake of material. In addition to electroporation, transfection can be carried out by various methods of introducing foreign DNA into a cell.

One method is transfection by calcium phosphate (see e.g. Nature Methods 2, 319-320). HEPES-buffered saline solution (HeBS) containing phosphate ions is combined with a calcium chloride solution containing the DNA to be transfected. When both solutions are combined, a fine precipitate of the positively charged calcium and the negatively charged phosphate will form, binding the DNA to be transfected on its surface. The suspension of the precipitate is then added to the cells to be transfected (usually a cell culture grown in a monolayer). Many materials have been used as carriers for transfection, among them (cationic) polymers, liposomes and nanoparticles (see e.g. U.S. Pat. No. 5,948,878, Felgner et al., 1987; Martien et al., 2008). Such methods use e.g. highly branched organic compounds, so-called dendrimers, to bind the DNA. A very efficient method is the inclusion of the DNA to be transfected in liposomes capable of fusing with the cell membrane, releasing the DNA into the cell. For eukaryotic cells, lipid-cation based transfection is more typically used, because the cells are more sensitive. Another method is the use of cationic polymers such as DEAE-dextran or polyethylenimine. The negatively charged DNA binds to the polycation and the complex is taken up by the cell via endocytosis. Transfection can also be effected with the gene gun, as described above.

Other methods of transfection include nucleofection, heat shock, magnetofection and transfection reagents such as Lipofectamine™, Dojindo HilyMax, Fugene, jetPEI™, Effectene or DreamFect™.

It is preferred that the introduction of the nucleic acid is stable, i.e. that it stably resides in the nucleus. If the nucleic acid introduced does not itself encode a selectable marker which provides the cell with a selection advantage, such as a resistance towards a certain herbicide, toxin or antibiotic and if the nucleic acid introduced is not stably incorporated into the nucleus, incorporation can be promoted by co-transformation with another nucleic acid encoding such a selectable marker.

A nucleic acid, if it encodes a protein, introduced into the nucleus is generally translated in the cytosol (cytosolic expression). The present method aims at increasing cytosolic expression, whereas expression can also take place in cell organelles such as mitochondria or chloroplasts which have their own genome encoding organelle-specific genes. The latter expression type is, however, preferably not envisaged by the present invention.

A “selectable marker” as used in connection with the present invention denotes genetic information which provides the cell with a selection advantage as compared to other cells which do not contain said selectable marker. Examples of selectable markers include expression products of resistance genes to toxins, herbicides or antibiotics or of genes encoding proteins restoring prototrophy of the cell for a specific organic compound or allowing for growth under adverse conditions.

In the context with the present method, selectable markers are used which are responsive to a selecting agent and wherein the level of expression of said selectable marker is proportional to the level of phenotypic responsiveness to the selecting agent. A selecting agent provides a disadvantage to cells which do not have a gene encoding a selectable marker responsive to said selecting agent which is able to neutralize the effect of said selecting agent. “Phenotypic responsiveness” denotes the extent of a detectable reaction of the cell expressing a selectable marker to a selecting agent. In other words, the higher the expression of said selectable marker in the cell, the more clearly detectable is the expression of said selectable marker in the presence of selecting agent.

In a preferred embodiment, responsiveness to the selecting agent is resistance to the selecting agent. In this embodiment, the selectable marker confers a resistance. Said resistance is preferably detectable by the survival or growth of the cell in the presence of said selecting agent. This applies in particular if the selecting agent is an antibiotic, toxin or herbicide, i.e. when the selectable marker confers resistance to said antibiotic, toxin or herbicide. Accordingly, the higher the expression of said selectable marker conferring a resistance in the cell, the higher is the dose of selecting agent such as an antibiotic, toxin or herbicide applicable to said cell without inhibiting its growth or killing it.

Commonly introduced and expressed selectable markers in mammals which confer a resistance are, for example, dihydrofolate reductase (dhfr) conferring resistance to cycloguanil, guanine hypoxanthine phosphoribosyltransferase (gpt) conferring resistance to mycophenolic acid, neomycin phosphotransferase II conferring resistance to neomycin or a hygromycin inactivating kinase conferring resistance to hygromycin B. Typical selectable marker genes for fungal cells include, for example, the benanomycin resistance (benA) gene, kanamycin resistance gene, G418 resistance gene and bleomycin resistance gene. Plant cell selectable marker genes are those expressing oligomycin-resistant ATP synthase (oliC), hygromycin B resistance, kanamycin resistance, G418 (geneticin) resistance, phleomycin/bleomycin resistance, emetin resistance, paromomycin resistance or BAR which confers resistance to the herbicide BASTA.

As described above, the selectable marker can be the expression product of a gene encoding a protein restoring prototrophy for an organic compound, also referred to as prototrophy restoring gene. In this case, the selectable marker introduced enables the cell to synthesize said compound by itself so that it is no longer or less dependent on the external supply of said compound with the medium. Accordingly, a prototrophy restoring gene as used in the present invention is a gene encoding an expression product, i.e. the selectable marker, which reduces or preferably abolishes the dependency of the host cell on external supply of an organic compound by facilitating its synthesis in the cell. Selection for cells expressing said prototrophy restoring gene is carried out by culturing said cells on/in medium not containing said compound. Only cells expressing said prototrophy restoring gene will grow. The expression product of said gene may be a constituent of a synthesis pathway and the product produced by said constituent may have to be further processed in order to obtain the organic compound otherwise externally supplied. Prototrophy restoring genes commonly applied to plant or fungal cells are e.g. those expressing proteins conferring arginine prototrophy, tryptophan prototrophy, uridine prototrophy or genes enabling for nitrate or sulphate utilization. If the selectable marker is the expression product of a prototrophy restoring gene, the selecting agent is the medium in which the cell is cultivated and which does not contain the respective organic compound. Responsiveness in that case is expressed e.g. in growth rates of the cell. Thus, the higher the expression of the selectable marker, the higher the growth rate of the cell in the absence of the respective compound.

For some prototrophy restoring genes, the amount of expression product sufficient to result in prototrophy is very low. Accordingly, it is more laborious to distinguish cells expressing said prototrophy restoring selectable marker at a low level from those that express it at a high level. In order to facilitate said distinction, such a prototrophy restoring gene can be co-introduced together with a nucleic acid encoding a reporter gene the detectability of which is proportional to its expression level. Accordingly, in this embodiment, the selectable marker according to the invention is composed of the auxotrophy gene and the reporter gene.

“Reporter genes” are genes the expression products of which are measurable over background level and detectable with commonly applied and straightforward methods. Said expression products are also termed “reporters”. Reporter genes can be generally used to gain information about the expression behaviour of cells, about the expression and localization of other genes if fused to the reporter gene, or about the activity of the promoter controlling the reporter gene. Certain expressed reporters are visually detectable such as fluorescent or phosphorescent proteins. Others, such as luciferase, convert a substrate into a visually detectable product which e.g. emits bioluminescence. The above reporters are detectable in the cell. Further reporters are those for which specific binding molecules such as antibodies exist. These can be detected by methods well-known in the art applying such binding molecules such as immunoprecipitation or Western blotting. Reporters or reporter genes in the form of nucleic acids can be detected by Southern or Northern blotting. However, detection of these reporters cannot take place within the cell but they have to be liberated from the cell. Examples of suitable reporter genes in this embodiment of the present invention are fluorescent or phosphorescent proteins described in detail further below or those mediating bioluminescence. An exemplary combination of a selectable marker and a reporter gene for the organism Chlamydomonas fulfilling the above prerequisites consists of the Arg gene and GFP or YFP.

The term “fluorescent protein” or “fluorescent (poly)peptide” refers to (poly)peptides or proteins emitting fluorescent light upon excitation at a specific wavelength. A variety of fluorescent proteins can be used in the present invention. One group of such fluorescent proteins includes Green Fluorescent Protein isolated from Aequorea victoria (GFP), as well as a number of GFP variants, such as cyan fluorescent protein, blue fluorescent protein, yellow fluorescent protein, etc. (Zhang et al., 2002 ; Zimmer, 2002). Color-shift GFP mutants have emission colors blue to yellow-green, increased brightness, and photostability (Tsien, 1998). One such GFP mutant, termed the Enhanced Yellow Fluorescent Protein, displays an emission maximum at 529 nm. Additional GFP-based variants having modified excitation and emission spectra (U.S. Patent Application 200201231), enhanced fluorescence intensity and thermal tolerance (U.S. Patent Application 20020107362A1; U.S. Patent Application 20020177189A1), and chromophore formation under reduced oxygen levels (U.S. Pat. No. 6,414,119) have also been described.

Phosphorescence is a specific type of photoluminescence related to fluorescence. Unlike fluorescence, a phosphorescent material does not immediately re-emit the radiation it absorbs. The slower time scales of the re-emission are associated with “forbidden” energy state transitions in quantum mechanics. As these transitions occur less often in certain materials, absorbed radiation may be re-emitted at a lower intensity for up to several hours. Examples for phosphorescent proteins are phosphorescent metalloporphyrins.

Bioluminescence is the production and emission of light by a living organism as the result of a chemical reaction during which chemical energy is converted to light energy. Bioluminescence may be naturally generated by symbiotic organisms carried within larger organisms. Adenosine triphosphate (ATP) is involved in most instances. The chemical reaction can occur either inside or outside the cell. Exemplary proteins causing bioluminescence are firefly luciferase, Renilla luciferase and Gaussia luciferase.

Selection conditions for cells expressing a selectable marker depend on the properties of the selectable marker applied. If an antibiotic resistance has been introduced into a cell, it will be cultured in/on a medium containing the respective antibiotic in concentrations sufficient to ensure selection of transformed cells.

As described above, if a prototrophy restoring gene is introduced alone or in combination with a reporter gene, the cell will be cultivated on a medium not containing the organic compound synthesized by the gene product of said prototrophy restoring gene.

In the present method, selection is effected for cells with a detectable expression of said selectable marker. Depending on the selectable marker, detectability is expressed as survival and/or growth of the cells or the expression of a sufficient amount of the selectable marker to be detected otherwise, e.g. by an altered phenotype of the cells. Regarding the example of an antibiotic resistance, detectable expression means that different clones obtained after introduction of the selectable marker are each subjected to different concentrations of the respective antibiotic. Clones showing resistance expressed in growth and/or survival at a high concentration of said antibiotic are selected in step (b) and later on in step (d). In the case of prototrophy restoring genes, clones grown on a medium without the respective organic compound and containing the reporter gene are selected in steps (b) and later on in step (d).

“Propagating” denotes increasing the number of cells starting from one or more cells, in this case those obtained in step (b), by culturing them in/on an appropriate medium containing the selecting agent. In general, a clone obtained after step (b) becomes detectable/visible and/or distinguishable after propagation of said clone, e.g. in the form of colonies. Accordingly, a propagation step is inherently part of the selection step (b). However, under certain circumstances, e.g. if a high number of cells is needed or desired, an additional step of propagating may be carried out.

“Mutagenizing the cell” denotes the process of randomly altering the genetic information contained in the cell. Depending on the method used, mutagenesis results in cells altered to a smaller or larger extent. In the context of the present invention, mutagenesis targets the genetic information contained in the cell nucleus since the process believed to be responsible for the poor expression of transgenes in cells, e.g. a gene silencing mechanism, is assumed to be located in the nucleus rather than in the cell organelles. Mutagenesis can be effected, for example, through chemical mutagens, by irradiation or by genetic methods.

Chemical mutagens are defined as compounds that increase the frequency of some types of mutations. They vary in their potency, their reactivity with DNA, their general toxicity, and the likelihood that the type of chemical alteration they introduce into the DNA will be corrected by a repair system.

Several classes of chemical mutagens are described: Base analog mutagens are chemicals that mimic normal bases and are used by the DNA replication system. Their essential property is that they base-pair with two different bases thus causing mutations because of their lack of consistency in base-pairing. An example is 5-bromo-deoxyuridine (5BU), which can exist in two tautomeric forms: typically it exists in a keto form (T mimic) that pairs with A, but it can also exist in an enol form (C mimic) that pairs with G. Each base analog mutagen will continue to mutagenize with time because of its constant likelihood of mispairing. Accordingly, subsequent rounds of replication are required for any mutation to be generated since this requires “mispairing” during replication. Further, it takes another round of replication before the mutation is stabilized, that is, before both strands of DNA have the mutant information. This is termed “mutation fixation”. Until mutation fixation occurs, the mismatch repair system can still recognize and remove the inappropriate base.

Alkylators react directly with certain bases and thus do not require active DNA synthesis in order to act but still require DNA synthesis in order to cause a “fixed mutation”. They are very commonly used because they are powerful mutagens in nearly every biological system. Examples of alkylators include ethyl methane sulfonate (EMS), methyl methane sulfonate (MMS), diethylsulfate (DES), and nitrosoguanidine (NTG, NG, MNNG). These mutagens tend to prefer G-rich regions, reacting to form a variety of modified G residues, the result often being depurination. Some of these modified G residues have the property of inducing error-prone repair although mispairing of the altered base might also be possible. This stimulation of error-prone repair allows all sorts of mutation types to occur as a result of these mutagens, though base substitutions are by far the most frequent. It also appears that alkylated bases can mispair during replication.

Another chemical mutagen is nitrous acid that causes oxidative deamination of particular bases. It converts adenine to hypoxanthine (which now pairs with C), cytosine to uracil (which now pairs with A) and finally guanine to xanthine (which still continues to pair with C). Unlike the above mutagens, nitrous acid alters a base directly to a “miscoding” form and thus does not require subsequent DNA synthesis for its effect.

Yet another class of chemical mutagens, the so-called “ICR” compounds (heterocyclic nitrogen mustards), induce frameshift mutations which require DNA synthesis in order to cause mutations. They apparently mutagenize by “intercalating” between adjacent bases, perhaps making synthesis/repair systems think there is another base at that position.

Mutagenesis by irradiation is preferably conducted by UV light which generates primarily cyclobutane dimers and pyr(6-4)pyo photo products at adjacent pyrimidine bases. Other irradiation-based mutagenesis techniques utilize, for example, X-ray or fast neutron bombardment.

Genetic mutagenesis techniques include, for example, random insertional mutagenesis (e.g, with plasmids or Agrobacterium T-DNA constructs) and transposon mutagenesis e.g., Azpiroz-Leehan (1997), Bouchez (1998), Zhang (2003), Ostergaard (2004), Alonso (2006).

Alternatively to actively mutagenizing the cells, mutagenesis can also be accomplished by allowing for the appearance of spontaneous mutations in the cells. The mutation rate in cells is usually very low due to various DNA repair systems present in the cells. However, certain environmental conditions are suitable to rise the rate of spontaneous mutations. Such conditions are e.g. a rise in the temperature during culturing or exposure to certain cell or tissue culture media (a phenomenon also known as ‘somaclonal variation’).

After mutagenesis, selection is carried out for a cell displaying increased expression of the selection marker as compared to the cell obtained in step (b). In the case of an antibiotic resistance as selectable marker, this means that a higher dose of selecting agent can be applied to a clone obtained after mutagenesis as to that obtained in step (b) without inhibiting its growth or killing it. An increased expression preferably means an increase of at least 10%, preferably at least 25%, more preferred at least 50%, even more preferred at least 100%, such as at least 200%, at least 300% or at least 400% and most preferably at least 500% of expressed gene product as compared to the cell obtained in step (b). For prototrophy restoring genes, a clone grown on a medium without the respective organic compound and displaying a higher detectable expression of the reporter gene as compared to other clones, wherein the expression is higher than that detected in step (b) is selected in step (d).

The present invention for the first time discloses an effective method for generating eukaryotic cells with an increased expression of transgenes. Methods aiming at the same have been proposed numerous times in the literature, but, however, have not yielded comparable results. The present method is based on the interplay of a selectable marker transformed into cells of interest and a following step of mutagenizing the cells. Only the combination of these steps enables for the selection of cells exerting a higher expression of said selectable marker as compared to naturally occurring cells based on the inactivation of the process responsible for suppressing the expression of transgenes in the cell. The cells obtained with the present method provide valuable research tools. Furthermore, they may serve as efficient expression hosts for a number of proteins or nucleic acids such as biopharmaceuticals or biofuel components the recombinant expression of which was so far not possible, since certain biologically active compounds could not be obtained, for example, due to the lack of post-translational modifications necessary to confer said activity.

As opposed to prokaryotic organisms, post-translational modifications are carried out in the cytosol of eukaryotic cells. Accordingly, the combination of efficient expression of transgenes and the possibility to effect the necessary modifications to the expression products to confer their biological activity provides a considerable improvement in the recombinant expression of certain proteins or nucleic acids. The method of the invention is especially applicable in cells which can be easily cultured and propagated such as plant cells or industrially applied yeasts. However, due to the increased expression, the method is valuable even for cells growing more slowly such as mammalian cells.

Finally, the strategy developed here to isolate cells exhibiting high transgene expression capacity is not only applicable to cells in which foreign protein accumulation levels are unsatisfactorily low but is also generally applicable to cells where no transgene expression problem exists, but where a further boost in expression capacity is desirable. The applicability of the strategy is also independent of a transcriptional versus post-transcriptional cause of poor transgene expression—the only difference would be that the screens would yield different kinds of mutants.

In a preferred embodiment, the cell is a plant cell, a fungal cell or a mammalian cell. Plant cells suitable in the present invention are, for example, alfalfa, Ethiopian mustard, potato, tobacco, Arabidopsis, Lemna, rice, banana, maize, soybean, cauliflower, mosses, such as Physcomitrella, tomato, corn, oilseeds, wheat or algae such as eukaryotic red and green algae, e.g. Chlamydomonas, Euglena and Chlorella. Suitable fungal cells are, for example, selected from strains belonging to the Aspergillus family, such as Aspergillus niger or Aspergillus oryzae, or Fusarium venenatum or yeasts such as Saccharomyces cerevisiae, Saccharomyces diastaticus or Pichia pastoris or other industrially applicable yeast species. Commonly applied mammalian expression hosts include but are not restricted to human Hela, HEK 293, H9, SH-EP1 and Jurkat cells, mouse NIH3T3 and C2C12 cells, Cos 1, Cos 7 and CV1, quail QC1-3 cells, mouse L cells, Syrian golden baby hamster kidney (BHK) cells and Chinese hamster ovary (CHO) cells.

The cells can be unicellular organisms or derived from a multicellular organism. In the latter case, e.g. cells of a tissue or tissue explants can be taken and cultivated. Furthermore, the cells obtained with the method of the present invention may form/develop into a multicellular transgenic organism, e.g. a transgenic plant, fungus or a non-human transgenic animal, if the appropriate cells such as non-human embryonal stem cells are chosen as starting material.

In another preferred embodiment, the cells are Chlamydomonas cells.

Although transgenic clones are readily obtained at high frequency upon transforming Chlamydomonas cells, it has been notoriously difficult to identify clones that express the foreign gene of interest to reasonably high levels (Fuhrmann et al., 1999; Schroda et al., 2000). The molecular reasons for the very low efficiency of transgene expression in Chlamydomonas are not understood. Possible mechanisms include the presence of unorthodox epigenetic gene silencing activities and/or an exceptionally compact chromatin structure that usually does not permit active transcription of transgenes.

Use of specialized promoters (Schroda et al., 2000; Fischer and Rochaix, 2001) and resynthesis of the transgene's coding region to adjust its codon usage to that of the highly GC-rich nuclear genome of Chlamydomonas (Fuhrmann et al., 1999; Fuhrmann et al., 2004; Shao and Bock, 2008) alleviated the problem to some extent in some cases, but no general solution has been found.

In an attempt to overcome the limitations of transgene expression observed in several eukaryotic organisms on the example of the model organism Chlamydomonas reinhardtii, a genetic screening procedure destined to allow the selection of Chlamydomonas mutants that express introduced transgenes to high levels in the cytosol was developed. As evident from the appended examples, algal expression strains could be isolated that accumulate fluorescent proteins expressed from the nuclear transgenes to as much as 0.2% of the total soluble protein. The strains have the potential to solve the severe transgene expression problems in Chlamydomonas and to greatly expand the toolbox for Chlamydomonas cell and molecular biology.

In the present invention, dedicated strains of the model alga Chlamydomonas reinhardtii were developed that express introduced foreign genes, i.e. trangenes, to high levels. These strains will help to overcome the arguably most serious limitation in both basic and applied research with Chlamydomonas. The strains were selected by their capability to express a native Chlamydomonas gene, an antibiotic-resistant allele of the RPS14 gene (CRY1-1), to high levels. However, different selectable markers are just as suitable in the present method.

Importantly, the strains also express introduced heterologous transgenes to very high levels, such as the genes for the fluorescent reporter proteins GFP and YFP as shown in the examples. Furthermore, expression was shown to be promoter-independent as no substantial differences could be observed when expressing GFP under the control of the PsaD and the RBCS2 promoters. This now opens up the possibility to apply a whole set of previously unusable techniques to Chlamydomonas, including subcellular localization analyses and in vivo protein-protein interaction studies using FRET, BiFC and similar methods. Together with the recently completed genome sequence of Chlamydomonas, the availability of these techniques is expected to greatly accelerate post-genomics research in green algae. In addition, the overexpression of endogenous genes, which often provides valuable information about gene functions, should now become much less troublesome.

There is also a strongly growing interest in exploiting Chlamydomonas for biotechnological purposes, for example, as a production system for biofuels (Happe et al., 2002; Kruse et al., 2005) and for the contained and cost-effective expression of biopharmaceuticals, an area commonly referred to as molecular farming (Franklin and Mayfield, 2004; Walker et al., 2005). As, thus far, all these applications have been greatly hampered by the very low transgene expression levels attained in Chlamydomonas, the strains described here will also help to overcome one of the most serious bottlenecks in algal biotechnology.

Previous attempts to utilize Chlamydomonas as an expression host were based on the adaptation of the codons of transgenes introduced to the codon usage of Chlamydomonas. Interestingly, the efficiency of transgene expression in the strains obtained in the present invention was no longer dependent on codon usage. A codon-optimized GFP and a non-codon-optimized YFP gene could be expressed to comparably high levels.

Cytosolic expression of proteins as achieved in the present invention is particularly advantageous as compared to that in cell organelles because post-translational modifications which are often prerequisite for the biological activity of an expressed protein are exclusively effected in the cytosol. On the contrary, organelles which have most likely developed from bacteria do not possess many of these mechanisms. Accordingly, even if high expression of some proteins could be achieved in cell organelles such as the chloroplast of Chlamydomonas in the prior art, a major part of useful proteins will not be expressed in a biologically active form.

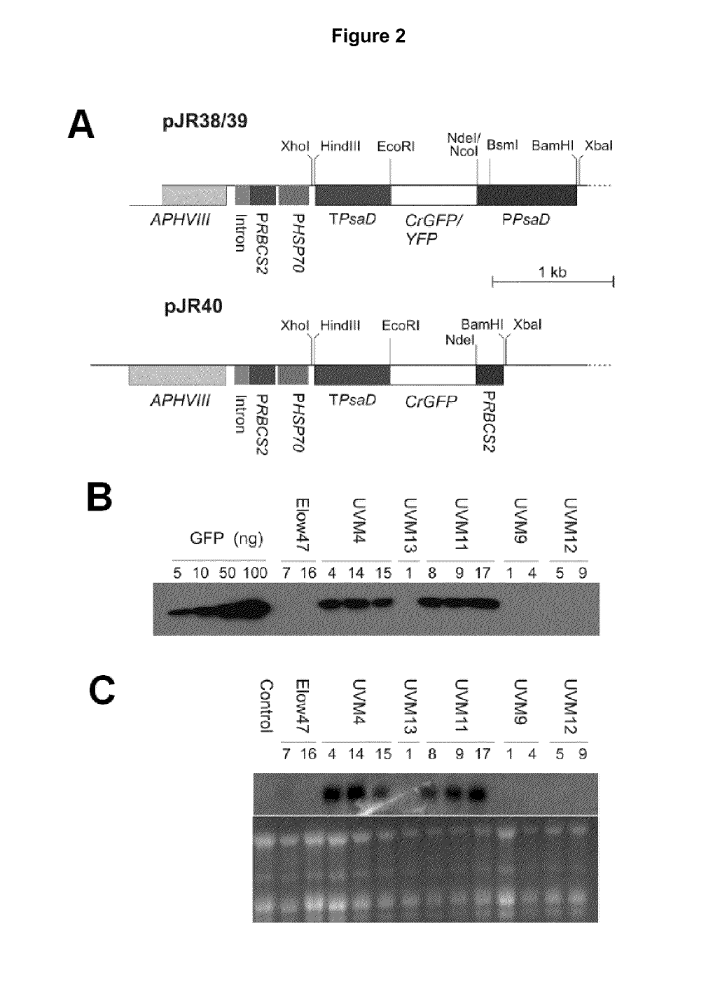

Moreover, all independently generated transgenic clones produced in the present invention displayed comparably high foreign protein accumulation levels (FIG. 2B). This suggests that, in contrast to wild-type strains, expression levels are no longer much dependent on the insertion site in the genome.

In addition to poor expression of foreign genes, instability of transgene expression has been frequently observed in Chlamydomonas (Fuhrmann et al., 1999). Clones that initially showed some transgene expression, lost it later for unknown reasons. At least for the three transgenes experimentally tested so far (CRY1-1, GFP and YFP), no evidence of instability of transgene expression in the strains produced in the present invention could be seen.

The prior art could not quantify the expression yield of transgenes expressed in the cytosol of a Chlamydomonas cell since the yield was generally too low to be quantified. Accordingly, with the method of the present invention, a major drawback of the utilization of Chlamydomonas as expression host can be overcome by providing strains exerting an increased expression capacity. Depending on the transgene expressed, the yield may be higher or lower than 0.2% of the total soluble protein of the cell.

It is also possible to transfer the advantageous properties of a Chlamydomonas strain, such as a Chlamydomonas rheinhardtii strain, obtained by applying the method of the present invention to other Chlamydomonas strains by conventional crossing methods well known in the art. Exemplary strains include cell wall-deficient and cell wall-containing strains of Chlamydomonas rheinhardtii and interfertile Chlamydomonas species, like Chlamydomonas smithii. This technique can be expanded to other eukaryotic cells.

Accordingly, in a further preferred embodiment, the method of the invention further comprises the step of crossing cells selected for in step (e) with interfertile eukaryotic cells and selecting for non-parent cells displaying said increased expression of said selectable marker as observed in step (e).

In another preferred embodiment, the responsiveness is resistance and the selectable marker confers a resistance.

In a more preferred embodiment, the selection in steps (b) and (d) is for a cell showing resistance to the highest concentration of said antibiotic.

Due to the different mechanism of action of the antibiotics applied and depending on the cell used, a “higher concentration” or the “highest concentration” of an antibiotic may differ. The actual “higher concentration” or “highest concentration” for a specific antibiotic and for the specific cell used can be determined by routine methods known to the skilled person. For example can rough screens with a wide range of concentrations applied to the culture medium indicate in which highest tolerable range said concentration is to be found. More detailed screens around the initial concentration determined will yield the exact “higher or highest concentration”. For this aspect of the method of the present invention, the difference between the antibiotic concentrations applicable to the cell prior to and after mutagenizing is determined. Commonly obtained highest applicable concentrations of antibiotics can vary by several orders of magnitude. For kanamycin, for example, concentrations observed are usually in the range of 10 to 1000 mg/l.

In a more preferred embodiment, the resistance gene is the CRY1-1 gene. As described above, said CRY1-1 gene is an allele of the RPS14 gene (CRY1-1) the expression of which confers resistance to emetin.

In another preferred embodiment, the method of the present invention further comprises (a)′ introducing a nucleic acid encoding a selectable marker responsive to a selecting agent different than that applied in step (a) into the cells prior to step (b) and (b)′ selecting for responsiveness to said selectable marker with said selecting agent preferably after step (a) and prior to step (b).

This embodiment takes into account that the selection in step (b) with only a selectable marker as defined above may be more laborious since the levels of expression observed or the differences in expression are very low. This holds true e.g. for Chlamydomonas, if selection with specific antibiotics such as emetine is carried out because expression of the respective selectable marker, i.e. the CRY1-1 gene, is sometimes difficult to observe. Accordingly, to facilitate appropriate selection or in order to enhance the yield of cells selected in step (b), a nucleic acid encoding a further selectable marker can be introduced into the cells and an additional selection step for cells responsive to said selectable marker is carried out.

In a more preferred embodiment, the cells are auxotrophic for a compound, wherein the selectable marker is an auxotrophy gene encoding a protein restoring prototrophy for said compound and wherein step (b)′ comprises selecting for the restoration of prototrophy for said compound after step (a) and prior to step (b).

In an even more preferred embodiment, the cell is a Chlamydomonas cell which is auxotrophic for the Arg7 gene.

Auxotrophy is the opposite of prototrophy which has been described above and denotes the inability of an organism to synthesize a particular organic compound required for its growth (as defined by IUPAC). In the context of genetic methods, a cell is said to be auxotrophic if it carries a mutation that renders it unable to synthesize an essential compound. For example a yeast mutant in which a gene of the uracil synthesis pathway is inactivated is a uracil auxotroph. Such a strain is unable to synthesize uracil and will only be able to grow if uracil can be taken up from the environment. Another example is a Chlamydomonas mutant, wherein the gene expressing an enzyme for arginine synthesis is inactivated. Accordingly, arginine has to be taken up from the environment of the cell, e.g. the culture medium.

In a further preferred embodiment, the transgene is under the control of the PsaD or the RBCS2 promoter.

In another preferred embodiment, mutagenesis is carried out by irradiation, preferably UV-irradiation, chemical mutagenesis or genetic mutagenesis all of which have been discussed above.

In another preferred embodiment, the method further comprises repeating steps (d) and (e) after step (e). This embodiment serves to obtain cells with further increased expression of the selectable marker as compared to cells having undergone steps (d) and (e) only once. Steps (d) and (e) are repeated at least once resulting in an exemplary sequence of step (a), (b), (c), (d), (e), (d), (e), . . . of the method according to this preferred embodiment. Steps (d) and (e) may be repeated until no further increase in expression as compared to the preceding round of steps (d) and (e) can be observed.

In another preferred embodiment, the method further comprises inactivating or removing the selectable marker introduced in step (a) and optionally that in step (a)′ after step (e).

Inactivating the selectable marker aims at restoring the original responsiveness of the cell to the selecting agent. This can be accomplished by crossing out the previously introduced gene encoding the selectable marker, i.e. removing it, or by deleting said gene completely or partially so that it does not yield a functional expression product. These methods are well known to the skilled person and include, for example, marker elimination by site-specific recombination (Ebinuma et al., 2001).

This embodiment serves to restore the cells' original sensitivity to a selecting agent to enable for the potential re-use of said selectable marker for introducing transgenes of interest, if necessary or desired.

In another preferred embodiment, the method further comprises (f) introducing a nucleic acid molecule encoding a transgene of interest and optionally a selectable marker responsive to a selecting agent into the cells obtained in step (e); and (g) assaying for expression of said transgene or a compound modulated by the expression product of said transgene in said cell in the presence of said selecting agent.

It is preferred that step (f) is carried out after step (e).

The term “compound modulated by the expression product of said transgene” refers to any compound within the cell, the presence or amount of which is effected by said expression product. Alternatively, this term includes embodiments wherein a compound is changed in its nature by the activity or by the presence of said expression product. This compound may, e.g., be an educt or a product. Changes in the amount or presence include embodiments wherein the transgene expression product is an siRNA, shRNA or miRNA as well as wherein the expression product is an anti-sense construct etc. Examples of changes in the nature of the product include those where said expression product is an enzyme and the compound is a substrate of the enzyme (or the turnover product of enzyme activity, if the compound is the product).

In this as well as in other embodiments of the present invention comprising the introduction of nucleic acid molecules, the transgene and the selection marker, if applicable, can be contained in one or more nucleic acids. If this introduction, e. g. in the form of transformation, is carried out using one nucleic acid, a commonly applied form is a plasmid carrying both the transgene and the selectable marker gene to be introduced. Otherwise, more than one plasmid each carrying one gene may be used. Also mixtures of plasmids containing one or more genes each can be used.

The one or more genes introduced can be reporter genes the general application of which is described further below. If the transgene introduced is a reporter gene, this particular embodiment may serve as a confirmation that the cell obtained in step (d) is capable of expressing a transgene apart from the selectable marker introduced in step (a) in high amounts. Particularly suitable reporters for this purpose are fluorescent or phosphorescent proteins or proteins mediating bioluminescence.

A selectable marker is co-introduced if no expression of the transgene introduced alone can be obtained. In this case, the selectable marker is preferably different from that introduced in step (a) to increase the chance of obtaining cells expressing the transgene. However, the selectable marker can also be the same as that introduced in step (a) which is preferably the case if the latter has been inactivated in the cells used.

As has been briefly described above, the method of the present invention does not only enable for the increased expression of a specific selectable marker introduced into the cell but, more importantly, for the expression of transgenes in general. Such transgenes comprise those that produce industrially important proteins (such as enzymes), e.g. for the production of biofuels, biopharmaceuticals etc. In other terms, this embodiment of the present invention provides a production plant for the preparation of a large variety of expression products. If the expression product of the transgene itself is the product of interest, then higher values of this expression product are desired, preferably at least 0.001% of total (soluble) protein. If the compound modulated by the expression product for said transgene is assayed for, then the amount of expression product of the transgene may be much lower. For example, in the case of siRNA molecules, a few molecules per se may suffice in order to obtain reduced or abolished expression of the target gene. Similarly, if the transgenic expression product is an enzyme, a lower number of molecules may suffice in order to obtain the desired product which arises from a substrate of a compound to be assayed for (if the compound is the product). This holds particularly true, if the desired compound is a biopharmaceutical. Likely, the present method results in the inactivation of the general mechanism responsible for an impaired expression of transgenes in eukaryotic cells and not only in the selective expression of the selectable marker introduced.

In an even more preferred embodiment, the method further comprises assaying for the presence of the complete transcription unit of the nucleic acid molecule encoding said transgene in the nucleus of the cell obtained in step (f) after introduction of said transgene.

A complete transcription unit includes the coding sequence of the transgene as well as optionally regulatory elements present in the nucleic acid originally introduced into the cell such as a promoter. If a selectable marker was co-transformed in the same transcription cassette, the coding region of said marker as well as the above regulatory elements for said marker may also need to be present.

This embodiment serves to verify that a lack of detectable expression of a transgene results from the transgene which was not or not fully incorporated into the nucleus of the cell. Methods of assessing for the presence of the nucleic acid encoding a transgene in the nucleus of the cell are well-known to the skilled person and include PCR, RT-PCR, Northern blotting or Southern blotting.

Optionally, for comparative purposes, said nucleic acid encoding the transgene and optionally a selectable marker, can also be introduced into the cells obtained in step (b). In this case, assaying for the presence of the complete transcription unit of the nucleic acid molecule encoding said transgene and optionally the selectable marker may serve as a positive control that the nucleic acid was incorporated if, as expected from cells obtained in step (b) expression of the transgene cannot be observed.

Techniques for the determination of the presence of a nucleic acid include, but are not limited to PCR and its various modifications such as qRT-PCR (also referred to as Real Time RT-PCR). PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed for example in a 50 μl reaction mixture containing 5 μl of 10× PCR buffer with 1.5 mM MgCl2, 200 μM of each deoxynucleoside triphosphate, 0.5 μl of each primer (10 μM), about 10 to 100 ng of template DNA and 1 to 2.5 units of Taq DNA polymerase. The primers for the amplification may be labeled or unlabeled. DNA amplification can be performed, e.g., with a model 2400 thermal cycler (Applied Biosystems, Foster City, Calif.): 2 min at 94° C., followed by 30 to 40 cycles consisting of annealing (e. g. 30 s at 50° C.), extension (e. g. 1 min at 72° C., depending on the length of DNA template and the enzyme used), denaturing (e. g. 10 s at 94° C.) and a final annealing step at 55° C. for 1 min as well as a final extension step at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. However, the person skilled in the art knows how to optimize PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template.

The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746,121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT Reaction can be performed, for example, in a 20 μl reaction mix containing: 4 μl of 5× AMV-RT buffer, 2 μl of Oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70° C. for 15 minutes to allow for reverse transcription. The reaction temperature is then raised to 95° C. for 1 minute to denature the RNA-cDNA duplex. Next, the reaction temperature undergoes two cycles of 95° C. for 15 seconds and 60° C. for 20 seconds followed by 38 cycles of 90° C. for 15 seconds and 60° C. for 20 seconds. Finally, the reaction temperature is held at 60° C. for 4 minutes for the final extension step, cooled to 15° C., and held at that temperature until further processing of the amplified sample. Any of the above mentioned reaction conditions may be scaled up according to the needs of the particular case.

A Southern blot is used to check for the presence of a DNA sequence in a DNA sample. Southern blotting combines agarose gel electrophoresis for size separation of DNA with methods to transfer the size-separated DNA to a filter membrane for probe hybridization (Southern, 1975).

Northern blotting is a technique used to study gene expression. It takes its name from the similarity of the procedure to the Southern blot procedure used to study DNA, with the key difference that, in the northern blot, RNA, rather than DNA, is the substance being analyzed by electrophoresis and detection with a hybridization probe (Alwine et al., 1977).

Protocols on how to perform any of the two blotting techniques are well-known to the skilled person.

In a different aspect, the present invention relates to a method of producing a compound of interest in a cell produced with the method of the present invention comprising (a) introducing a nucleic acid encoding (i) the compound of interest which is a protein or an RNA; or (ii) a protein necessary to synthesize said compound of interest; and optionally a selectable marker responsive to a selecting agent into said cell; (b) expressing said protein in the cell; and (c) isolating the compound of interest produced.

When a compound of interest is a protein or an RNA, increased expression of said protein or RNA already leads to the product of interest. As soon as expression is completed, the protein or RNA may be isolated from the cells. In the case that the compound of interest is not a protein or RNA but a substance which can be synthesized by one or more proteins or RNAs, the expression of said one or more proteins or RNAs leads to their accumulation and correspondingly to the synthesis of the compound of interest.

Compounds of interest can be e.g. pharmaceuticals, including vaccines, toxins, antibiotics, antibodies or therapeutic enzymes; biofuel components; diagnostic compounds or chemicals such as polymers or metabolites.

A selectable marker is co-introduced if no expression of the transgene introduced alone can be obtained. In this case, the selectable marker is preferably different from that introduced in step (a) of the method of the invention for generating eukaryotic cells to increase the chance of obtaining cells expressing the transgene. However, the selectable marker can also be the same as that introduced in step (a) of said method which is preferably the case if the latter marker has been inactivated in the cells used.

This method is applicable with cells obtainable with the method of the present invention which were not yet transformed with a transgene, e. g. according to step (f).

A large number of suitable methods exist in the art to produce polypeptides (or fusion proteins) in appropriate hosts. If the host is a unicellular organism such as a unicellular plant organism or a cell derived from a multicellular organism such as a mammal or insect, plant or fungus the person skilled in the art can revert to a variety of culture conditions. Conveniently, the produced protein is harvested from the culture medium, lysates of the cultured organisms or from isolated (biological) membranes by established techniques. In the case of a multicellular organism, the host may be a cell which is part of or derived from a part of the organism, for example said host cell may be the harvestable part of a plant. A preferred method involves the recombinant production of protein in hosts as indicated above. For example, nucleic acid sequences comprising the transgene(s) of interest can be synthesized by PCR and inserted into an expression vector. Subsequently a cell produced with the method of the present invention may be transformed with the expression vector. Thereafter, the cell is cultured to produce/express the desired protein(s), which is/are isolated and purified.

Commonly applied methods of cultivating Chlamydomonas for expression purposes are described in the appended examples and can furthermore be retrieved by the skilled person, e.g., from Harris (1989) or Franklin and Mayfield (2004). Similarly, methods of cultivating other organisms or cells for expression purposes are well-known, e.g. for higher plants (Potrykus I, 1991; McElroy and Brettell, 1994; Hansen and Wright, 1999; Bilang et al, 1999; Ma et al., 2003).

“Isolating the compound” refers to the separation of the compound produced during or after expression of the nucleic acid introduced. After disintegrating the cells, various separation methods are known in the art. In the case of proteins or peptides as expression products, said proteins or peptides, apart from the sequence necessary and sufficient for the protein to be functional, may comprise additional N- or C-terminal amino acid sequences. Such proteins are sometimes also referred to as fusion proteins. Additional amino acid sequences can be tags facilitating the purification of said proteins. Exemplary tags are a 6× Histidin tag (SEQ ID NO: 11) or a GST-tag. A Tap-tag enables for multi-step purification of proteins in complex with their interaction partners.

The term “fusion protein” generally refers to chimeric proteins consisting of sequences derived from at least two different proteins or (poly)peptides. Fusion may be performed by any technique known to the skilled person, as long as it results in the in frame fusion of the nucleic acid molecules encoding the components of the fusion proteins described herein. Fusion of the components may be effected in any order. Conventionally, the generation of a fusion protein from two or more separate (poly)peptides or domains is based on the “two-sided splicing by overlap extension” described in (Horton et al., 1989). The fragments coding for the single (poly)peptides are generated in two separate primary PCR reactions. The inner primers for the primary PCR reactions contain a significant, approximately 20 bp, complementary region that allows the fusion of the two domain fragments in the second PCR. Alternatively, the coding regions may be fused by making use of restriction sites which may either be naturally occurring or be introduced by recombinant DNA technology.

The components of the fusion protein utilized throughout the present invention may be separated by a linker. A linker can be a peptide bond or a stretch of amino acids comprising at least one amino acid residue which may be arranged between the components of the fusion proteins in any order. Such a linker may in some cases be useful, for example, to improve separate folding of the individual domains or to modulate the stability of the fusion protein. Moreover, such linker residues may contain signals for transport, protease recognition sequences or signals for secondary modification. The amino acid residues forming the linker may be structured or unstructured. Preferably, the linker may be as short as 1 amino acid residue or up to 2, 3, 4, 5, 10, 20 or 50 residues. In particular cases, the linker may even involve up to 100 or 150 residues.

Protein isolation and purification can be achieved by any one of several known techniques; for example and without limitation, ion exchange chromatography, gel filtration chromatography and affinity chromatography, high pressure liquid chromatography (HPLC), reversed phase HPLC, and preparative disc gel electrophoresis. Protein isolation/purification techniques may require modification of the proteins of the present invention using conventional methods. For example, a histidine tag can be added to the protein to allow purification on a nickel column. Other modifications may cause higher or lower activity, permit higher levels of protein production, or simplify purification of the protein.

In a preferred embodiment of the methods of the present invention, the nucleic acid introduced has been adapted to the codon usage of said cell.

An amino acid is specified on the nucleic acid level by triplets of nucleotides referred to as codons. Due to four existing nucleotides, there are 64 possible triplets to recognize 20 amino acids plus the translation termination signal. Because of this redundancy, all but two amino acids are encoded by more than one triplet. Different organisms often show particular preferences for one of the several codons that encode the same given amino acid referred to as “codon usage” in the present invention. It is generally acknowledged that codon preferences reflect a balance between mutational biases and natural selection for translational optimization. Optimal codons in fast-growing microorganisms, like Escherichia coli or Saccharomyces cerevisiae, reflect the composition of their respective genomic tRNA pool. It is thought that optimal codons help to achieve faster translation rates and high accuracy. As a result of these factors, translational selection is expected to be stronger in highly expressed genes, as is indeed the case for the above-mentioned organisms. In other organisms that do not show high growing rates or that present small genomes, codon usage optimization is normally absent, and codon preferences are determined by the characteristic mutational biases seen in that particular genome. Examples of this are Homo sapiens and Helicobacter pylori. Organisms that show an intermediate level of codon usage optimization include Drosophila melanogaster, Caenorhabditis elegans or Arabidopsis thaliana.

It is not clear at present whether codon usage drives tRNA evolution or vice versa. At least one mathematical model has been developed where both codon usage and tRNA expression co-evolve in a feedback fashion (i.e., codons already present in high frequencies drive up the expression of their corresponding tRNAs, and tRNAs normally expressed at high levels drive up the frequency of their corresponding codons!), however this model does not seem to yet have experimental confirmation. Different factors have been proposed to be related to codon usage bias, including gene expression level (reflecting selection for optimizing the translation process by tRNA abundance), % G+C composition (reflecting horizontal gene transfer or mutational bias), GC skew (reflecting strand-specific mutational bias), amino acid conservation, protein hydropathy, transcriptional selection, RNA stability, optimal growth temperature and hypersaline adaptation (Ermolaeva, 2001; Lynn et al., 2002).

In the field of bioinformatics and computational biology, many statistical methods have been proposed and used to analyze codon usage bias (Comeron and Aguade, 1998). Methods such as the ‘frequency of optimal codons’ (Fop) and the ‘codon adaptation index’ (CAI) are used to predict gene expression levels, while methods such as the ‘effective number of codons’ (Nc) and Shannon entropy from information theory are used to measure codon usage evenness (World Wide Web Uniform Resource Locator codonw.sourceforge.net/Indices.html; Suzuki et al., 2004). Multivariate statistical methods, such as correspondence analysis and principal component analysis, are widely used to analyze variations in codon usage among genes (Perriere and Thiolouse, 2002). There are many computer programs to implement the statistical analyses enumerated above, including CodonW (World Wide Web Uniform Resource Locator codonw.sourceforge.net/), and G-language GAE (World Wide Web Uniform Resource Locator g-language.org/wiki/).

Exemplary practical approaches of using codon-adapted nucleic acids for expression in Chlamydomonas are disclosed in Mayfield et al. (2003) and Franklin et al. (2002).

In a different aspect, the present invention relates to a eukaryotic cell produced by the method of the invention.

In a further aspect, the present invention relates to a kit comprising (a) a cell obtainable by the method of the invention and optionally a vector optimized for protein expression in said cell; or (b) the cell of the invention.

The various components of the kit may be packaged in one or more containers such as one or more vials. The vials may, in addition to the components, comprise preservatives or buffers for storage.

Said vector optimized for protein expression in said cell is preferably comprised in the kit of the invention if the cell obtainable by the method of the present invention was not yet transformed with a transgene.

In a further aspect, the present invention relates to a method of detecting the expression and/or localization of a protein in the cell generated with the method of the invention, comprising (a) expressing a nucleic acid encoding said protein fused to a reporter in said cell; or (a)′ expressing a nucleic acid encoding said protein which is a reporter in said cell; and (b) detecting the expression and/or localization of said reporter in said cell.

This method is applicable if the cell generated by the method of the present invention was not yet transformed with a transgene.

A “reporter gene” has already been defined elsewhere in this application. Certain genes are chosen as reporters because the characteristics they confer on organisms expressing them are easily identified and measured, or because they are selectable markers. Reporter genes may be used to determine whether the gene of interest has been taken up by or expressed in the cell or organism population. It is important to use a reporter gene that is not natively expressed in the cell or organism under study, since the expression of the reporter is being used as a marker for successful uptake of the gene of interest. Commonly used reporter genes that induce visually identifiable characteristics have already been described above and usually involve fluorescent or phosphorescent proteins or enzymes catalyzing reactions whose products can be readily detected (e.g., luciferases, β-galactosidase, β-glucuronidase).

Reporter genes are commonly used in transformation such as transfection assays. Reporter genes used in this way are normally expressed under their own promoter independent from that of the introduced gene of interest; the reporter gene can be expressed constitutively or inducibly with an external intervention such as the introduction of an agent switching on expression. As a result, the reporter gene's expression is independent of the gene of interest's expression, which is an advantage when the gene of interest is only expressed under certain specific conditions or in tissues that are difficult to access. In the case of selectable-marker reporters conferring e.g. a resistance to an antibiotic or a prototrophy restoring gene, the transfected cells can be grown on a substrate that contains the respective antibiotic or does not comprise the auxotrophic substance.

Another application for reporter genes is in gene expression assays for the expression of the gene of interest, which may produce a protein that has little obvious or immediate effect on the cell culture or organism. In these cases the reporter is directly attached to the gene of interest to create a gene fusion. The two genes are under control of the same promoter and are transcribed into a single messenger RNA molecule. The mRNA is then translated into protein. In these cases it is important that both proteins be able to properly fold into their active conformations and interact with their substrates despite being fused. In building the DNA construct, a segment of DNA coding for a flexible polypeptide linker region is usually included so that the reporter and the gene product of interest only minimally interfere with one another.

Reporter genes can furthermore be used to assay for the activity of a particular promoter in a cell or organism. In this case there is no separate “gene of interest”; the reporter gene is simply placed under the control of the target promoter and the activity of the reporter gene product is quantitatively measured. The results are normally reported relative to the activity under a “consensus” promoter known to induce strong gene expression.

In context of the present invention, a gene encoding a protein of interest may be fused to a reporter gene. Fusion proteins have been described further above.

Detecting the expression may be effected on different levels. On the transcription level, mRNAs expressed form the gene may be detected by methods such as PCR, RT-PCR or Northern blotting described above. On the translation level, proteinaceous expression products may be detected in vitro using e.g. Western blotting or immunoprecipitation, all well-known to the skilled person. Detection in the (living) cell can be established if the reporter gene possesses a property enabling for non-invasive detection. Exemplary properties are fluorescence, phosphorescence or bioluminescence which can be detected using appropriate microscopes or photometers.

Detecting the localization refers to detecting the localization of the expression product of the gene introduced into the cell. If the gene of interest is fused to a reporter gene, its localization can be determined via detecting the localization of the expression product of the reporter gene. Suitable reporter genes, the localization of which can also be detected in vivo are fluorescent, phosphorescent or bioluminescent proteins described elsewhere in this application. Detection of the above exemplary reporters can be effected by the experimenter or by specialized software known to the skilled person (e.g. ImageJ co-localization plug-ins, World Wide Web Uniform Resource Locator rsb.info.nih.gov/ij/).

In a different aspect, the present invention relates to an in vitro method for detecting protein-protein interactions in the cell generated with the method of the present invention comprising (a) expressing in said cell (i) a first nucleic acid encoding a fusion protein comprising a (poly)peptide of interest fused to a detectable marker and (ii) a second nucleic acid encoding a fusion protein comprising a (poly)peptide suspected of interacting with said first (poly)peptide fused to a different detectable marker and (b) detecting the localization of both detectable markers; wherein a co-localization of both detectable markers in the cell is indicative of an interaction.

This method is preferably applicable if the cell obtainable by the method of the present was not yet transformed with a transgene.

The term “protein-protein interactions” refers to the specific interaction of two or more proteinaceous compounds, i.e. poly(peptides) or proteins. Specific interaction is characterized by a minimum binding strength or affinity. Binding affinities for specific interactions generally reach from the pM to the mM range and also largely depend on the chemical environment, e.g. the pH value, the ionic strength, the presence of co-factors etc. In the context of the present invention, the term particularly refers to protein-protein interactions occurring under physiological conditions, i.e. in a cell.

The term “detectable marker” refers to the property of the fused protein to be visualizable without interfering with the living cell in which the marker is expressed. The group of detectable markers thus constitutes a subgroup of reporters. Exemplary detectable markers emit radiation such as fluorescence (e.g., GFP and its color variants), phosphorescence or bioluminescence (e.g., luciferases).

“Co-localization of both detectable markers in the cell” denotes the localization of two different emissions at the same site of the cell. Co-localization is detected as soon as two proteins interact with each other. Co-localization is e.g. detected as the partial or complete spatial overlap of emission from two different detectable (poly)peptides in the cell. Detection can be effected by the experimenter or by specialized software known to the skilled person (e.g. ImageJ co-localization plug-ins, World Wide Web Uniform Resource Locator rsb.info.nih.gov/ij/). The detectable marker is preferably a fluorescent or phosphorescent protein.

In this embodiment as well as in other embodiments wherein fluorescence is determined, detection is preferably carried out using a fluorescence microscope.

A fluorescence microscope is a light microscope used to study properties of organic or inorganic substances using the phenomena of fluorescence and phosphorescence instead of, or in addition to, reflection and absorption. The specimen is illuminated with light of a specific wavelength (or wavelengths) which is absorbed by the fluorophores, causing them to emit longer wavelengths of light (of a different color than the absorbed light). The illumination light is separated from the much weaker emitted fluorescence through the use of an emission filter. Typical components of a fluorescence microscope are the light source (Xenon or Mercury arc-discharge lamp), the excitation filter, the dichroic mirror (or dichromatic beamsplitter), and the emission filter. The filters and the dichroic mirror are chosen to match the spectral excitation and emission characteristics of the fluorophore used to label the specimen. Most fluorescence microscopes in use are epi-fluorescence microscopes (i.e.: excitation and observation of the fluorescence are from above (epi) the specimen). These microscopes have become an important part in the field of biology, opening the doors for more advanced microscope designs, such as the confocal laser scanning microscope (CLSM) and the total internal reflection fluorescence microscope (TIRF). These technologies are well known to the skilled person.

The present invention also envisages methods for the detection of protein-protein interactions using commonly applied techniques such as FRET, BiFC or FRAP in the cell produced by the method of the present invention.

Förster resonance energy transfer (FRET, also referred to as fluorescence resonance energy transfer if both molecules used are fluorescent) describes an energy transfer mechanism between two chromophores. A donor chromophore in its excited state can transfer energy by a nonradiative, long-range dipole-dipole coupling mechanism to an acceptor chromophore in close proximity (typically <10 nm) termed “Förster resonance energy transfer” (FRET) (Förster, 1949).

In fluorescence microscopy, fluorescence confocal laser scanning microscopy, as well as in molecular biology, FRET is a useful tool to quantify molecular dynamics in biophysics and biochemistry, such as protein-protein interactions, protein-DNA interactions, and protein conformational changes (Lakowicz, 1999). For monitoring the complex formation between two molecules, one of them is labeled with a donor and the other with an acceptor, and these fluorophore-labeled molecules are mixed. When they are dissociated, the donor emission is detected upon the donor excitation. On the other hand, when the donor and acceptor are in proximity (1-10 nm) due to the interaction of the two molecules, the acceptor emission is predominantly observed because of the intermolecular FRET from the donor to the acceptor. For monitoring protein conformational changes, the target protein is labeled with a donor and an acceptor at two loci. When a twist or bend of the protein brings the change in the distance or relative orientation of the donor and acceptor, FRET change is observed. If a molecular interaction or a protein conformational change is dependent on ligand binding, this FRET technique is applicable to fluorescent indicators for the ligand detection.

FRET studies are scalable: the extent of energy transfer is often quantified from the milliliter scale of cuvette-based experiments to the femtoliter scale of microscopy-based experiments. This quantification can be based directly (sensitized emission method) on detecting two emission channels under two different excitation conditions (primarily donor and primarily acceptor). However, for robustness reasons, FRET quantification is most often based on measuring changes in fluorescence intensity or fluorescence lifetime upon changing the experimental conditions (e.g. a microscope image of donor emission is taken with the acceptor being present. The acceptor is then bleached, such that it is incapable of accepting energy transfer and another donor emission image is acquired.) An alternative way of temporarily deactivating the acceptor is based on its fluorescence saturation. Exploiting polarisation characteristics of light, a FRET quantification is also possible with only a single camera exposure.

The most popular FRET pair for biological use is a cyan fluorescent protein (CFP)-yellow fluorescent protein (YFP) pair. Both are color variants of green fluorescent protein (GFP). While labeling with organic fluorescent dyes requires troublesome processes of purification, chemical modification, and intracellular injection of a host protein, GFP variants can be easily attached to a host protein by genetic engineering.

A limitation of FRET is the requirement for external illumination to initiate the fluorescence transfer, which can lead to background noise in the results from direct excitation of the acceptor or to photobleaching. To avoid this drawback, Bioluminescence Resonance Energy Transfer (or BRET) has been developed. This technique uses a bioluminescent luciferase (typically the luciferase from Renilla reniformis) rather than CFP to produce an initial photon emission compatible with YFP.

Bimolecular fluorescence complementation (BiFC) is a method of viewing the association of proteins inside living cells (Hu et al., 2002). The method is based on the fluorescent properties of some proteins such as GFP and its variants. When the fluorescent protein is split into N and C-terminal halves, the molecule does not produce fluorescence. Fusing of each of the two non-fluorescent fragments to two putative interacting partners leads to restoration of fluorescence within a cell by reconstituting the split flurophore. This fluorescence is detected via fluorescence microscopy, which can be recorded by a mounted camera. The advantage of the BiFC method over other methods of visualizing protein-protein interactions is that it gives an indication of interaction, as well as cellular localization of the complex. BiFC can be used as an alternative to FRET, or it can complement FRET by its possibility of screening protein-protein interactions and their modulators through combination with other techniques.

The FRAP (fluorescence recovery after photobleaching) method denotes an optical technique capable of quantifying the diffusion and mobility of fluorescently labelled probes. This technique provides a great utility in biological studies of protein binding and is commonly used in conjunction with fluorescent proteins, where the studied protein is fused to a fluorescent protein. When excited by a specific wavelength of light (typically with a laser beam), the protein will fluoresce. When the protein that is being studied is produced in fusion with the fluorescent protein, then the fluorescence can be tracked. After photodestruction of the fluorescent protein (typically with a strong/intense laser pulse), the kinetics of fluorescence recovery in the bleached area provide information about strength of protein interactions, organelle continuity and protein trafficking that prevents or slows down the exchange of bleached and unbleached fluorescent proteins. This observation has most recently been exploited to investigate protein binding.

The figures show:

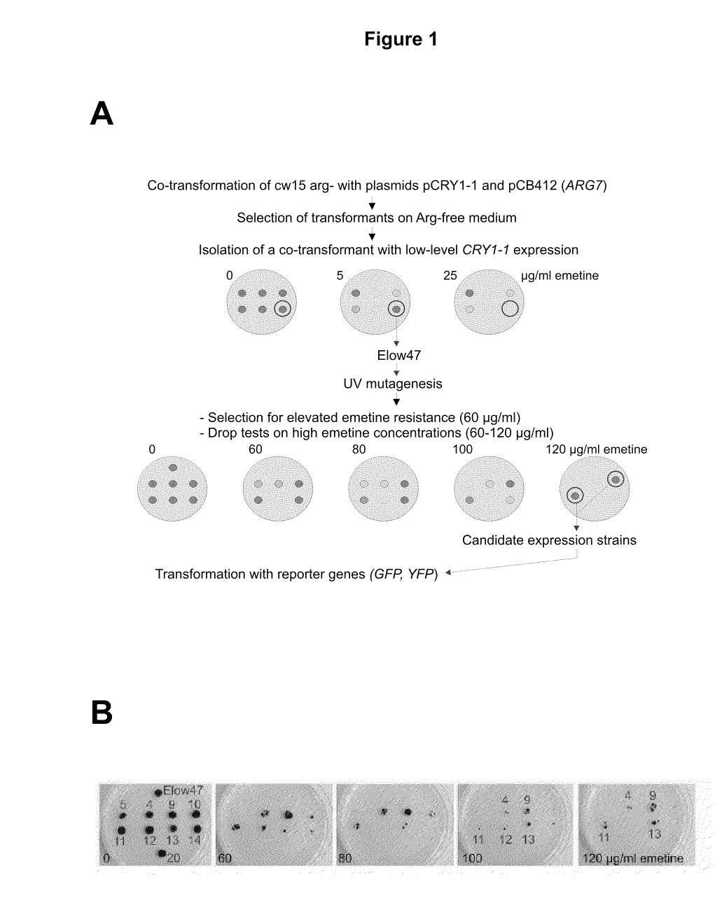

FIG. 1. Genetic screen to select Chlamydomonas strains that express nuclear transgenes to high levels. (A) Overview of the experimental strategy designed to generate Chlamydomonas expression strains (B) Identification of candidate strains from the mutagenesis experiment which show high-level resistance to the ribosome-inhibiting drug emetine. The five strains displaying the highest tolerance to emetine (UVM4, UVM9, UVM11, UVM12 and UVM13) were chosen for further analysis.

FIG. 2. Identification of Chlamydomonas strains that express transgenes to high levels. (A) Transgene expression vectors constructed in this study. Vector pJR38 contains a synthetic GFP gene that was codon-optimized for Chlamydomonas reinhardtii (CrGFP; 10) and is driven by the PsaD promoter (PPsaD; Fischer and Rochaix, 2001). The plasmid also contains the APHVIII gene as a selectable marker that confers resistance to paromomycin (Sizova et al., 2001) and is driven by a hybrid promoter consisting of fused expression elements from the HSP70A gene (PHSP70) and the RbcS2 gene (PRBCS2). Vector pJR39 harbors a ‘native’ YFP gene whose codon usage was not optimized. In vector pJR40, the synthetic GFP gene is under the control of the RbcS2 promoter. TPsaD: terminator from the PsaD gene (Fischer and Rochaix, 2001). (B) Analysis of GFP expression by western blotting using an anti-GFP antibody. 5 μg total soluble protein (TSP) of algal strains transformed with vector pJR38 were loaded per lane. For quantitation of GFP expression, a dilution series of purified GFP protein was included. All GFP transformants in strains UVM4 and UVM11 express the reporter gene to similarly high levels (˜0.2% of TSP). (C). Analysis of GFP mRNA accumulation by northern blotting. Samples of 3 μg of total RNA were separated by denaturing agarose gel electrophoresis, blotted and hybridized to a GFP-specific probe (upper panel). As a loading control, the ethidiumbromide stained gel is also shown (lower panel). Note that GFP mRNA accumulation levels are nearly identical in all transformed UVM4 and UVM11 clones. Control: untransformed UVM11 strain.

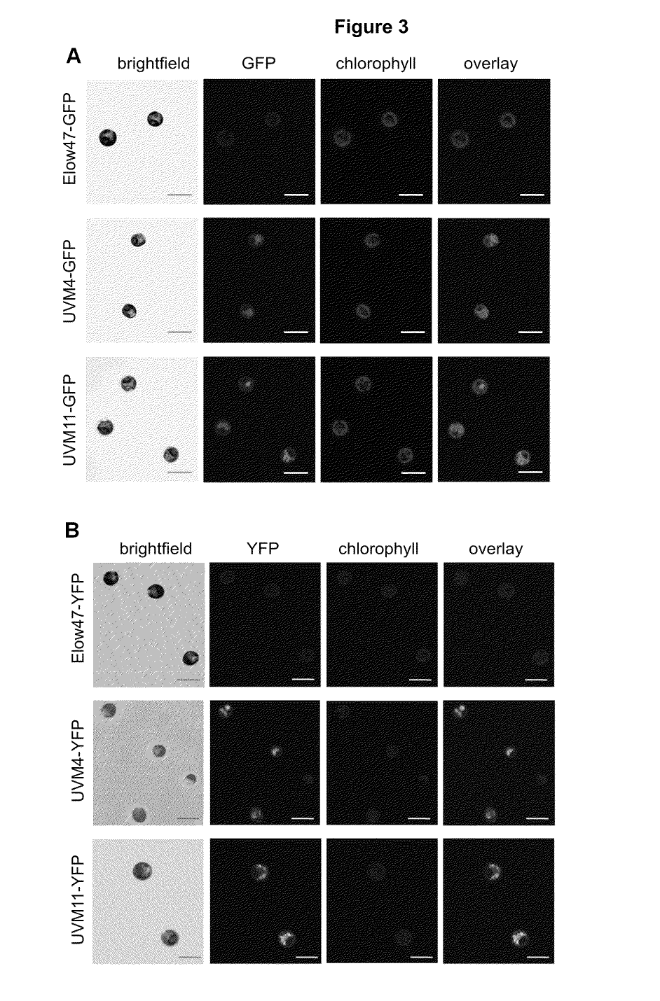

FIG. 3. Analysis of the accumulation of fluorescent reporter proteins GFP and YFP in by confocal laser-scanning microscopy. Fluorescence of UVM4, UVM11 and Elow47 cells transformed with pJR38 (GFP gene) or pJR39 (YFP gene) are shown. For comparison and subcellular localization, the bright-field image, the chlorophyll fluorescence and the overlay of reporter protein fluorescence and chlorophyll fluorescence are also shown. (A) Visualization of GFP expression in the cytosol. (B) Visualization of YFP expression. Scale bars: 10 μm.

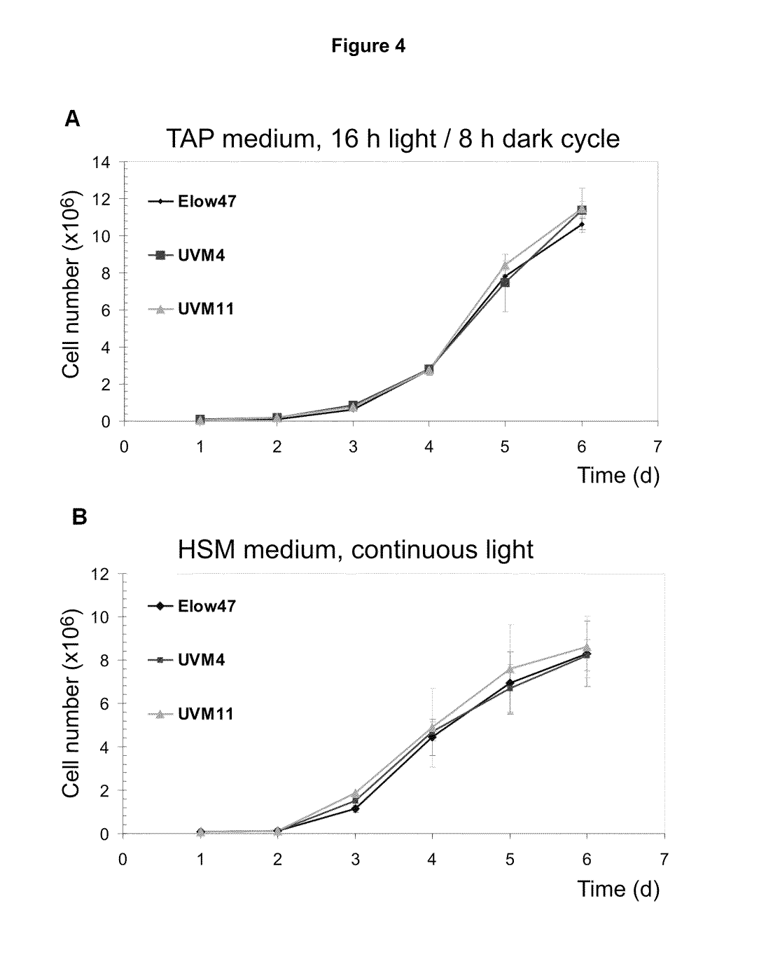

FIG. 4. Growth assays of Chlamydomonas expression strains UVM4 and UVM11 in comparison with the unmutagenized strain Elow47. (A) Growth curve recorded under photomixotrophic conditions. Cells were grown in TAP medium in a 16 h light/8 h dark cycle. (B) Growth under photoautotrophic conditions in HSM medium under continuous light. Cell numbers were determined with a cell counter at the time points indicated. All data represent averages of three biological replicas. The standard deviation is indicated by error bars.

The examples illustrate the invention.

### EXAMPLE 1

### Materials and Methods

Algal strains and culture conditions. The Chlamydomonas reinhardtii cell wall-deficient, arginine prototrophic strain (cw15 arg-, kindly provided by M. Schroda, University Freiburg, Germany) was used for transformation and was cultivated photomixotrophically either in liquid or on solid TAP medium (Harris, 1989) at 22° C. in a 16 h: 8 h day/night cycle (light intensity 50 μE m-2 s-1), unless otherwise stated. If required, arginine was added to the medium (100 μg ml-1). Strain Elow47 was obtained after co-transformation of cw15 arg- with pCRY1-1 (Nelson et al., 1994) and pCB412 (provided by C. F. Beck, University Freiburg, Germany) carrying the emetine resistance gene and the ARG7 gene, respectively. All UVM strains were obtained after UV light-induced mutagenesis of strain Elow47. For analysis of photoautotrophic growth, Chlamydomonas cells were cultivated in HSM liquid medium (Harris, 1989).

Construction of transformation vectors. The CrGFP sequence (Fuhrmann et al., 1999) was amplified with primers PCrGFPfw (5′-CATATGGCCAAGGGCGAGG-3′) (SEQ ID NO: 1) and PCrGFPrev (5′-GAATTCTTACTTGTACAGCTCGTCC-3′) (SEQ ID NO: 2) introducing NdeI and EcoRI sites at the 5′ and 3′ ends, respectively (restriction sites in italics). The GFP coding region was subsequently inserted as NdeI/EcoRI fragment into the similarly cut vector pGenD-PsaF (Fischer and Rochaix, 2001) resulting in plasmid pJR37. The GFP cassette from pJR37 was then inserted as XhoI/XbaI fragment into the similarly digested derivative of plasmid pSI103 which contains the APHVIII selectable marker gene (Sizova et al., 2001; kindly provided by Rachel Dent, UC Berkeley, USA) generating transformation vector pJR38 (FIG. 2A). Vector pJR39 containing YFP as reporter gene was constructed by amplification of the Venus YFP variant (Shyu et al., 2006) from a plasmid clone (kindly provided by Dr. Marc Lohse, MPI-MP) using the PCR primers PPsaD-YFPfw (5′-GTGCATTCTAGGACCCCACTGCTACTCACAACAAGCCCCATGGTGAGCAAGGGCG AGGAGC-3) (SEQ ID NO: 3) and PPsaD-YFPrev (5′-GAATTCTTACTTGATCAGCTCGTCCATGC-3′) (SEQ ID NO: 4) introducing a 5′ BsmI site (and an NcoI site containing the translational start codon) and a 3′ EcoRI site, respectively (restriction sites in italics). pJR39 was obtained by digestion of pJR38 with BsmI and EcoRI and replacing the GFP sequence by the YFP-containing BsmI/EcoRI fragment (FIG. 2A). To generate plasmid pJR40 (FIG. 2A), the RBCS2 promoter fragment was amplified from plasmid pBC1 (containing the HSP70A-RBCS2 hybrid promoter upstream of the APHVIII gene) with primers P5′RBCS2-BamHI (5′-AAGGATCCCCGGGCGCGCCAGAAGG-3′) (SEQ ID NO: 5) and P3′RBCS2-NdeI (5′-GGCGGCCATATGAAGATGTTGAGTG-3′) (SEQ ID NO: 6) introducing a 5′ BamHI site and a 3′ NdeI site (sequences in italics). The RBCS2 PCR fragment was then digested with BamHI and NdeI and ligated into pJR37 cut with the same enzymes, thereby replacing the PsaD promoter sequence upstream of GFP with the RBCS2 promoter. Finally, the RBCS2 promoter was excised as XbaI/NdeI fragment and cloned into the similarly digested vector pJR38 generating transformation vector pJR40 (FIG. 2A). DNA fragments were ligated into linearized plasmids with T4 DNA ligase (New England Biolabs, Frankfurt a.M., Germany) according to the manufacturer's instruction at 16° C. over night.

Transformation of C. reinhardtii. Nuclear transformation of C. reinhardtii was performed using the glass bead method (Kindle, 1990). Co-transformants of cw15 arg- with pCB412 (2.4 μg per transformation, linearized with EcoRI) and pCRY1-1(1 μg per transformation, linearized with EcoRI) were selected on arginine-free medium. UVM strains obtained after UV mutagenesis of Elow47 were transformed with vectors pJR38 (1 μg plasmid DNA linearized with KpnI), pJR39 (1 μg plasmid DNA linearized with KpnI) or pJR40 (0.8 μg plasmid DNA linearized with KpnI). Selection of transformants was performed on TAP medium containing 5-10 μg ml-1 paromomycin.

Emetine sensitivity tests. In order to isolate a strain suitable for mutagenesis and screening, CRY1-1/ARG7 co-transformants were tested for growth on TAP agar plates with increasing concentrations of emetine (0, 5, 25, 80 μg ml-1 emetine). Strain Elow47, which grew on 5 μg ml-1 emetine, but was sensitive to 25 μg ml-1 emetine (and thus displayed only very low expression of CRY1-1) was chosen for the subsequent mutagenesis experiment (FIG. 1A). Emetine sensitivity tests were also conducted after UV mutagenesis to analyze growth of mutants under elevated concentrations of emetine (up to 120 μg ml-1; FIG. 1).

UV mutagenesis. Elow47 cells were grown in 200 ml TAP medium to mid-log phase (cell density: 3.7×106 ml-1) and harvested by centrifugation (1700×g, 5 min). The algal pellet was resuspended in 2.5 ml fresh TAP medium and 8 samples of 250 μl each (equaling 7.4×107 cells) were plated on TAP agar plates containing 60 μg ml-1 emetine for direct selection of mutants exhibiting increased expression of the CRY1-1 transgene. Petri dishes were exposed to UV light in a distance of 13 cm to the UV lamp (ETX-20, 254 nm, 100 W, 50/60 Hz, LFT Labortechnik, Wasserburg, Germany) under sterile conditions for 1 min and directly transferred to the dark to minimize light induced cellular repair mechanisms. Following incubation in the dark over night, the cultures were incubated for 2 weeks under low light (5 μE m-2 s-1). Finally, UVM (UV mutagenesis) strains were analyzed by performing emetine sensitivity drop test. Strains showing growth at elevated concentrations of emetine (100-120 μg ml-1; FIG. 1) were further tested by transformation with different promoter-reporter gene constructs (FIG. 2A).

DNA isolation, purification and PCR. Genomic DNA from Chlamydomonas reinhardtii was extracted according to published methods (Schroda et al., 2001). For ligation of DNA fragments and isolation of hybridization probes, DNA was purified by agarose gel electrophoresis following extraction of the excised gel slices using the GFXTM PCR (DNA and Gel Band Purification) kit (GE Healthcare, Freiburg, Germany). PCR was performed according to standard protocols (1 min at 95° C., 90 s at 58° C.-66° C., 90 s at 72° C., 32 cycles). Transformants obtained with vector pJR38 were tested for presence of the entire GFP cassette by PCR. Primer pair APHVIIIrev (5′-CCTCAGAAGAACTCGTCCAACAGCC-3′) (SEQ ID NO: 7) and APHVIIIfw (5′-GGAGGATCTGGACGAGGAGCGGAAG-3′) (SEQ ID NO: 8) were used to amplify the 3′ end of APHVIII gene (360 bp product) and primers PPsaDrev (5′-CGAGCCCTTCGAACAGCCAGGCCG-3′) (SEQ ID NO: 9) and M13fw (5′-GTAAAACGACGGCCAGT-3′) (SEQ ID NO: 10) were used to amplify the 5′ end of the PsaD promoter in front of GFP (380 bp). Clones that yielded PCR products for both primer combinations were scored as positive for the full-length GFP cassette.

RNA extraction and northern blot analysis. Total cellular RNA was isolated from Elow47 and UVM strains transformed with pJR38 using the SV RNA Total Isolation kit (Promega, Mannheim, Germany) following the manufacturer's instructions. RNA samples was separated in 1% formaldehyde-containing agarose gels and blotted onto Hybond nylon membranes (GE Healthcare, Freiburg, Germany). Hybridizations were performed at 65° C. in Rapid-Hyb buffer (GE Healthcare) according to the manufacturer's protocol. The DNA template for generating a GFP-specific probe was amplified by PCR with primers PCrGFPfw (5′-CATATGGCCAAGGGCGAGG-3′) (SEQ ID NO: 1) and PCrGFPrev (5′-GAATTCTTACTTGTACAGCTCGTCC-3′) (SEQ ID NO: 2). Probes were radiolabeled by random priming using [α-32P]dCTP (GE Healthcare).

Protein isolation and western blot analyses. Total soluble protein extracts were prepared by resuspending pelleted Chlamydomonas cells in 200 μl lysis buffer (50 mM HEPES/KOH pH 7.5, 10 mM KAc, 5 mM MgAc, 1 mM EDTA, 1 mM DTT and 1× Protease Inhibitor Cocktail Complete; Roche, Mannheim, Germany) followed by disruption of cells by sonication (Sonifier®, W-250 D, G. Heinemann Ultraschall- and Labortechnik, Schwäbisch Gmünd, Germany; amplitude 10%, 15 s). Protein amounts were quantified according to the Bradford method (Roti®-Quant, Roth, Karlsruhe, Germany). Samples representing 5 μg of total soluble protein were denatured at 95° C. for 3 min, separated by denaturing SDS-polyacrylamide gel electrophoresis and transferred to PVDF (polyvinylidene difluoride) membranes (Hybond P, GE Healthcare, Freiburg, Germany) using the Trans-Blot® Electrophoretic Transfer Cell (Biorad, München, Germany) and standard transfer buffer (25 mM Tris/HCl, 192 mM glycine, pH 8.3). Immunobiochemical protein detection was carried out with a monoclonal anti-GFP primary antibody (Clontech, Saint-Germain-en-Laye, France) using the ECL detection system (GE Healthcare, Freiburg, Germany) and an anti-mouse secondary antibody (Sigma-Aldrich, Munich, Germany).

Microscopy. Reporter protein fluorescence was determined by confocal laser-scanning microscopy (TCS SP2; Leica, Wetzlar, Germany) using an argon laser for excitation (at 488 nm for GFP and 514 nm for YFP), a 490-510 nm filter for detection of GFP fluorescence, a 510-535 nm filter for detection of YFP fluorescence and a 630-720 nm filter for detection of chlorophyll fluorescence.

### EXAMPLE 2

### A Genetic Screen for Chlamydomonas Expression Strains

We assumed that there is a genetic basis to the transgene expression problem in Chlamydomonas, such as, an unusually tight chromatin structure or an epigenetic process that effectively silences incoming sequences. We further reasoned that, if this were the case, it should be possible to isolate mutants in which this transgene inactivation mechanism is defective. We therefore designed a genetic screen aiming at the selection of such mutants. The experimental strategy underlying the screen is outlined in FIG. 1A. To be able to directly select for efficient transgene expression, we chose a selectable marker gene whose expression level is proportional to the level of phenotypic resistance to a selecting agent. The CRY1-1 gene fulfills this criterion. It represents a mutant allele of the cytosolic ribosomal protein S14 which conditions insensitivity to the translational inhibitor emetine (Nelson et al., 1994). The more CRY1-1 protein (=mutant S14 protein) is made in the cell, the more efficiently the emetine-sensitive wild-type S14 protein is displaced from cytosolic ribosomes and the higher the emetine concentration the cell can tolerate. We introduced the CRY1-1 gene into the nuclear genome of an arginine-auxotrophic Chlamydomonas strain by co-transformation with the ARG7 selectable marker gene and selected for restoration of arginine prototrophy (FIG. 1A). Arginine-prototrophic strains were then assayed for co-transformation with the CRY1-1 gene. When tested on media with different concentrations of emetine, co-transformed strains displayed only low-level resistance to emetine (typically varying between 5 and 25 μg/ml), which is in line with poor transgene expression in the nuclear genome of Chlamydomonas. One such co-transformed strain, Elow47 (emetine resistant at low concentrations, strain number 47), was selected and subjected to UV light-induced mutagenesis (FIG. 1A). The rationale behind this strategy was that genetic inactivation of the transgene silencing mechanism operating in Chlaymdomonas would release the suspected transcriptional repression of the CRY1-1 gene, thereby facilitating growth on much higher emetine concentrations. Selection of the mutagenized cell population (6×108 mutagenized cells) for resistance to 60 μg/ml emetine indeed yielded clones that grew in the presence of high antibiotic concentrations. To determine the emetine resistance level in individual clones and identify those clones that display the strongest resistance, drop tests on media containing different concentrations of emetine were conducted with 22 clones that had shown clear growth on the primary selection plates in the presence of 60 μg/ml emetine (FIG. 1A,B, and data not shown). These experiments revealed substantial differences in the antibiotic resistance levels between strains indicating that different strains may carry different mutations that lead to elevated emetine resistance.

### EXAMPLE 3

### Identification of Strains that Efficiently Express Transgenes

Besides knock-out of the suspected transgene silencing mechanism, several alternative types of mutations could be responsible for the appearance of algal clones tolerating high concentrations of emetine. These include acquisition of emetine resistance-conferring point mutations in the endogenous RPS14 gene or mutations in the CRY1-1 cassette that allow for higher expression (e.g. mutations enhancing promoter strength, mRNA stability or translational efficiency). However, these undesired types of mutants can be easily distinguished from the sought-after ones, because they would not condition high expression of unrelated transgenes. We therefore tested five of the selected stains that displayed the highest level of emetine resistance (subsequently referred to as UVM4, UVM9, UVM11, UVM12 and UVM13; FIG. 1B) for their potential to express other transgenes to high levels. To this end, we constructed a transformation vector that harbors the most common fluorescent reporter gene, GFP. To exclude the possibility that the transgene expression capacity of the UVM stains was restricted to the promoter driving the CRY1-1 gene, the GFP coding region was placed under the control of expression signals that were different from the ones driving the CRY1-1 selectable marker gene in Elow47 (see Materials and Methods; FIG. 2A). As the expression of foreign genes in Chlamydomonas is believed to be dependent on the codon usage, we used a synthetic GFP gene whose sequence was adjusted to the codon usage in the nuclear genome of the alga (Fuhrmann et al., 1999).

We constructed a GFP expression vector (pJR38; FIG. 2A) containing the paromomycin resistance gene APHVIII (Sizova et al., 2001). All five candidate strains (UVM4, UVM9, UVM11, UVM12 and UVM13) were transformed with pJR38 and 17 to 20 paromomycin-resistant clones per construct were randomly chosen and analyzed for GFP expression by western blotting with a monoclonal anti-GFP antibody (FIG. 2B; Table 1). For three of the tested strains, UVM9, UVM12 and UVM13, not a single GFP-accumulating clone could be identified (FIG. 2B; Table 1) which is well in line with a large body of earlier work that had revealed great difficulties with foreign gene expression in Chlamydomonas (e.g., Fuhrmann et al., 1999; Schroda et al., 2000). However, two of the strains, UVM4 and UVM11, yielded GFP-expressing transformants at high frequency. 9 out of 17 analyzed paromomycin-resistant UVM4 clones and 9 out of 18 antibiotic-resistant UVM11 clones expressed GFP to high levels (FIG. 2B; Table 1). This tentatively suggested that strains UVM4 and UVM11 could represent mutants in which the epigenetic transgene inactivation mechanism has been knocked out. However, if this were indeed the case, one would expect 100% of the successfully transformed clones to express GFP. We therefore were interested in determining why approximately half of the UVM4 and UVM11 transformants did not exhibit detectable GFP expression (Table 1). Two alternative scenarios can potentially explain this finding: (i) a different epigenetic effect, such as position effects, prevented transgene expression in these clones or (ii) the clones do not contain the complete GFP cassette in their nuclear genome. As successfully transformed clones were only selected by their resistance to paromomycin (conferred by the APHVIII gene; FIG. 2A), it is not guaranteed that the GFP cassette is co-integrated in all antibiotic-resistant clones. To test for integration of the complete GFP cassette, PCR assays were conducted using a primer combination specific for the 5′ end of the promoter driving the GFP cassette and a second primer combination amplifying the 3′ end of the APHVIII gene (which is located immediately downstream of GFP; FIG. 2A). If both primer combinations yielded PCR products, we assumed that an intact GFP cassette was integrated into the genome. Interestingly, all those UVM4 and UVM11 transformants that did not show detectable GFP expression were negative in these PCR assays in that they lacked a complete GFP cassette. Thus, all transformants harboring the transgene also expressed it to high levels suggesting that strains UVM4 and UVM11 harbor mutations that have eliminated the epigenetic transgene inactivation mechanism. Interestingly, expression levels in all 18 UVM4 and UVM11 clones containing the GFP transgene were comparably high, suggesting that also no significant position effects operate in these strains. Quantitation of foreign protein accumulation against a dilution series of purified GFP (FIG. 2B) revealed that the GFP protein accumulated in the transformants to at least 0.2% of the total soluble protein. To our knowledge, this is the by far highest transgene expression level ever obtained by nuclear transformation in Chlamydomonas.

TABLE 1
GFP expression capacity in algal strains transformed
with a GFP gene cassette.

| | Number of | | | |
|---|---|---|---|---|
| | putative | | Number of | |
| | transformants₍₁₎ | Number of | GFP-positive | GFP-positive |
| | tested for | GFP | clones₍₃₎/ | clones |
| | GFP | expressing | clones tested | expressing |
| Strain | expression | clones₍₂₎ | by PCR | GFP [%] |
| UVM4-GFP | 17 | 9 | 9/17 | 100 |
| UVM9-GFP | 20 | 0 | 3/10 | 0 |
| UVM11-GFP | 18 | 9 | 9/18 | 100 |
| UVM12-GFP | 19 | 0 | 2/10 | 0 |
| UVM13-GFP | 19 | 0 | 3/10 | 0 |
| Elow47-GFP | 20 | 0 | 2/20 | 0 |

₍₁₎Paromomycin-resistant colonies prior to testing for integration of the full-length GFP cassette into the genome.
₍₂₎GFP expression was analyzed by both western blotting and fluorescence microscopy.
₍₃₎PCR tests for presence of the full-length GFP cassette in the genome.

We next wanted to determine whether or not foreign protein accumulation in the UVM4 and UVM11 transformants correlates with GFP mRNA accumulation. Northern blot experiments revealed that this was indeed the case. All clones showing high accumulation of the GFP protein also displayed comparably high accumulation of the GFP mRNA (FIG. 2C) suggesting a transcriptional nature of the transgene expression capacity of the two strains.

### EXAMPLE 4

### Development of New Fluorescent Reporters for Chlamydomonas

Having demonstrated the accumulation of GFP protein to high levels, we were interested to test the detectability of the protein by fluorescence microscopy. So far, GFP fluorescence could be seen in Chlamydomonas only with fusion proteins that were highly localized (e.g., in the flagella or the eyespot; Fuhrmann et al., 1999; Huang et al., 2007). In contrast, the GFP protein produced by the cassette in vector pJR38 is a free, unfused protein that should be present in the cytosol. Consistent with the high GFP accumulation levels measured by western blotting (FIG. 2B), GFP fluorescence was readily detectable in the cytosol of UVM4 and UVM11 strains (FIG. 3A). In contrast, Elow47 transformants harboring the pJR38 construct did not show fluorescence above background, again confirming that wild type-like Chlamydomonas strains do not express GFP to levels detectable by fluorescence microscopy (FIG. 3A).

Encouraged by the successful expression of GFP in our UVM4 and UVM11 strains, we set out to develop a second fluorescent reporter gene for Chlamydomonas. We chose YFP, because this is the second most frequently employed in vivo reporter of gene expression, which in addition, is also used for a variety of other cell biological applications, such as protein-protein interaction assays by bimolecular fluorescence complementation (BiFC) or fluorescence resonance energy transfer (FRET). We also used this second reporter to test whether or not codon usage adaptation has become dispensable in our expression strains UVM4 and UVM11. To this end, we inserted a non-codon-optimized YFP gene version into our transformation vector (pJR39 in FIG. 2A) and transformed the construct into UVM4, UVM11 and, as a control, Elow47. Analysis of transformed algal clones by fluorescence microscopy revealed that all UVM4 and UVM11 transformants carrying the complete YFP cassette showed bright yellow fluorescence in the cytosol, whereas none of the Elow47 transformants displayed any detectable above-background fluorescence (FIG. 3B and data not shown).

Finally, we wanted to confirm that our expression strains do not only express transgenes from the strong PsaD promoter (Fischer and Rochaix, 2001), but generally allow for efficient transgene expression in a promoter-independent manner. We, therefore, constructed an additional vector in which the GFP gene was driven by the promoter from the RBCS2 gene (pJR40; FIG. 2C) and transformed this cassette into the UVM4 and UVM11 strains. GFP accumulation was readily detectable by fluorescence microscopy in all transgenic clones harboring the complete GFP cassette (not shown) and fluorescence was similarly bright as with the PsaD promoter construct, suggesting that the two strains may represent generally applicable tools for achieving high-level transgene expression from the Chlamydomonas nuclear genome.

### EXAMPLE 5

### Wild-Type-Like Growth of the Chlamydomonas Expression Strains

It is conceivable that the epigenetic transgene inactivation mechanism that we apparently have knocked out in strains UVM4 and UVM11 serves some biological function. This function could either lie in the endogenous regulation of gene expression in Chlamydomonas or in some defense mechanism against invading nucleic acid sequences, such as viruses or intracellular bacterial pathogens. To test whether or not there is an important function of this epigenetic mechanism under standard conditions, we performed growth assays in which we compared the two strains with the non-mutagenized strain Elow47. We measured growth rates under both mixotrophic conditions (in TAP medium; FIG. 4A) and photoautotrophic conditions (in HSM medium; FIG. 4B) in either continuous light or a 16 h:8 h light/dark cycle. Under all conditions, the two mutant strains grew equally fast as the non-mutagenized control strain indicating that knock-out of the transgene-inactivating epigenetic mechanism does not confer a significant selective disadvantage.

### REFERENCES

Alonso J M, Ecker J R (2006) Moving forward in reverse: genetic technologies to enable genome-wide phenomic screens in Arabidopsis. Nature Rev Genet 7: 524-536.

Alwine, J. C., Kemp, D. J. and Stark, G. R. (1977). Method for detection of specific RNAs in agarose gels by transfer to diazobenzyloxymethyl-paper and hybridization with DNA probes. Proc. Natl. Acad. Sci. U.S.A. 74 (12): 5350-4.

An, G. (1987). Methods in Enzymol. 153: 292-305

Bebbington, C. R., Renner, G., Thomson, S., King, D., Abrams, D. and Yarranton, G. T. (1992). High-level expression of a recombinant antibody from myeloma cells using a glutamine synthetase gene as an amplifiable selectable marker. Biotechnology (N Y) 10:169-75.

Azpiroz-Leehan R, Feldman K A (1997) T-DNA insertion mutagenesis in Arabidopsis: going back and forth. Trends Genet 13: 152-156.

Bilang, R., Fütterer, J. and Sautter, C. (1999). Transformation of cereals. Genetic Engineering 21:113-157.

Bouchez D, Höfte H (1998) Functional genomics in plants. Plant Physiol 118: 725-732.

Bunch, T. A., Grinblat, Y. and Goldstein, L. S. (1988). Characterization and use of the Drosophila metallothionein promoter in cultured Drosophila melanogaster cells. Nucleic Acids Res. 16:1043-61.

Cerutti, H., Casas-Mollano, J. A. (2006) On the origin and functions of RNA-mediated silencing: from protists to man. Curr Genet 50: 81-99.

Comeron, J. M. and Aguade, M. (1998). An Evaluation of Measures of Synonymous Codon Usage Bias. Journal of Molecular Evolution 47(3):268-274.

Dent, R. M., Haglund, C. M., Chin, B. L., Kobayashi, M. C., Niyogi, K. K. (2005) Functional genomics of eukaryotic photosynthesis using insertional mutagenesis of Chlamydomonas reinhardtii. Plant Physiol 137: 545-556.

Ebinuma, H., Sugita, K., Matasunaga, E., Endo, S., Yamada and K., Komamine, A. (2001). Systems for the removal of a selection marker and their combination with a positive marker. Plant Cell Rep 20:383-392.

Ermolaeva, M. D. (2001). Synonymous codon usage in bacteria. Curr Issues Mol Biol. 3(4):91-7.

Feigner et al. (1987). Lipofection: a highly efficient, lipid-mediated DNA-transfection procedure, Proc Natl Acad Sci USA.

Fischer, N., Rochaix, J.-D. (2001) The flanking regions of PsaD drive efficient gene expression in the nucleus of the green alga Chlamydomonas reinhardtii. Mol Genet Genom 265: 888-894.

Förster T. (1948). Zwischenmolekulare Energiewanderung und Fluoreszenz, Ann. Physik 437: 55.

Franklin, S. E., Mayfield, S. P. (2004) Prospects for molecular farming in the green alga Chlamydomonas reinhardtii. Curr Op Plant Biol 7: 159-165.

Fuhrmann, M., Oertel, W., Hegemann, P. (1999) A synthetic gene coding for the green fluorescent protein (GFP) is a versatile reporter in Chlamydomonas reinhardtii. Plant J 19: 353-361.

Fuhrmann, M., Hausherr, A., Ferbitz, L., Schödl, T., Heitzer, M., Hegemann, P. (2004) Monitoring dynamic expression of nuclear genes in Chlamydomonas reinhardtii by using a synthetic luciferase reporter gene. Plant Mol Biol 55: 869-881.

Gietz, R. D. and Woods, R. A. (2002). Transformation of yeast by lithium acetate/single-stranded carrier DNA/polyethylene glycol method. Meth. Enzymol. 350: 87-96.

Happe, T., Hemschemeier, A., Winkler, M., Kaminski, A. (2002) Hydrogenase in green algae: Do they save the algae's life and solve our energy problems?. Trends Plant Sci 7: 246-250.

Harris, E. H. (1989) The Chlamydomonas Sourcebook. Academic Press, San Diego, Calif. Harris, E. H. (2001) Chlamydomonas as a model organism. Annu Rev Plant Physiol Plant Mol Biol 52: 363-406.

Hippler, M., Redding, K., Rochaix, J.-D. (1998) Chlamydomonas genetics, a tool for the study of bioenergetic pathways. Biochim Biophys Acta 1367: 1-62.

Horton, R. M., Hunt, H. D., Ho, S. N., Pullen, J. K. and Pease L. R. (1989). Engineering hybrid genes without the use of restriction enzymes: gene splicing by overlap extension. Gene. 77:61-8.

Hu, C. D., Grinberg, A. V. and Kerppola, T. K. (2005). Visualization of protein interactions in living cells using bimolecular fluorescence complementation (BiFC) analysis. Current Protocols in Cell Biology 21 (3) Wiley Interscience.

Huang, K., Diener, D. R., Mitchell, A., Pazour, G. J., Witman, G. B., Rosenbaum, J. L. (2007) Function and dynamics of PKD2 in Chlamydomonas reinhardtii flagella. J Cell Biol 179(3):501-14.

Kindle, K. L. (1990) High-frequency nuclear transformation of Chlamydomonas reinhardtii. Proc Natl Acad Sci USA 87: 1228-1232.

Kruse, O., Rupprecht, J., Bader, K.-P., Thomas-Hall, S., Schenk, P. M., Finazzi, G., Hankamer, B. (2005) Improved photobiological H2production in engineered green algal cells. J Biol Chem 280: 34170-34177.

Hansen, G. and Wright, M. S. (1999). Recent advances in the transformation of plants. Trends Plant Sci 4:226-231.

Lakowicz, J. R. (1999). Principles of Fluorescence Spectroscopy, Plenum Publishing Corporation, 2nd edition.

Lodha, M., Schroda, M. (2005) Analysis of chromatin structure in the control regions of the Chlamydomonas HSP70A and RBCS2 genes. Plant Mol Biol 59: 501-513.

Lynn, D. J., Singer, G. C. A. and Hickey, D. A. (2002). Synonymous codon usage is subject to selection in thermophilic bacteria. Nucleic Acids Research 30(19):4272-4277.

Ma, J. K.-C., Drake, P. M. W. and Christou, P. (2003). The production of recombinant pharmaceutical proteins in plants. Nature Genet 4:794-805.

McElroy, D. and Brettell, R. I. S. (1994). Foreign gene expression in transgenic cereals. Trends Biotechnol 12:62-68.

Martien, R., Loretz, B., Sandbichler, A. M. and Bernkop Schnürch, A. (2008). Thiolated chitosan nanoparticles: transfection study in the Caco-2 differentiated cell culture. Nanotechnology 19.

Merchant, S. S., Prochnik, S. E., Vallon, O., Harris, E. H., Karpowicz, S. J., Witman, G. B., Terry, A., Salamov, A., Fritz-Laylin, L. K., Maréchal-Drouard, L., Marshall, W. F., Qu, L.-H., Nelson, D. R., Sanderfoot, A. A., Spalding, M. H., Kapitonov, V. V., Ren, Q., Ferris, P., Lindquist, E., Shapiro, H., Lucas, S. M., Grimwood, J., Schmutz, J., Chlamydomonas Annotation Team, JGI Annotation Team, Grigoriev, I. V., Rokhsar, D. S., Grossman, A. R. (2007) The Chlamydomonas genome reveals the evolution of key animal and plant functions. Science 318: 245-251.

Murphy, G., Cockett, M. I., Ward, R. V. and Docherty, A. J. (1991). Matrix metalloproteinase degradation of elastin, type IV collagen and proteoglycan. A quantitative comparison of the activities of 95 kDa and 72 kDa gelatinases, stromelysins-1 and -2 and punctuated metalloproteinase (PUMP). Biochem J. 277 (Pt 1):277-9.

Nelson, J. A. E., Savereide, P. B., Lefebvre, P. A. (1994) The CRY1 gene in Chlamydomonas reinhardtii: Structure and use as a dominant selectable marker for nuclear transformation. Mol Cell Biol 14: 4011-4019.

Ostergaard L, Yanofsky M F (2004) Establishing gene function by mutagenesis in Arabidopsis thaliana. Plant J 39: 682-696.

Pedersen, L. B., Geimer, S., Rosenbaum, J. L. (2006) Dissecting the molecular mechanisms of intraflagellar transport in Chlamydomonas. Curr Biol 16: 450-459.

Perrière, G., Thioulouse, J. (2002). Use and misuse of correspondence analysis in codon usage studies. Nucleic Acids Res. 30(20):4548-55.

Potrykus, I. (1991). Gene transfer to plants: Assessment of published approaches and results. Annu Rev Plant Physiol Plant Mol Biol 42:205-225.

Remacle, C., Cardol, P., Cooseman, N., Gaisne, M., Bonnefoy, N. (2006) High-efficiency biolistic transformation of Chlamydomonas mitochondria can be used to insert mutations in complex I genes. Proc Natl Acad Sci USA 103: 4771-4776.

Rohr, J., Sarkar, N., Balenger, S., Jeong, B., Cerutti, H. (2004) Tandem inverted repeat system for selection of effective transgenic RNAi strains in Chlamydomonas. Plant J 40: 611-621.

Schiestl, R. H., Manivasakam, P., Woods, R. A. and Gietz, R. D. (1993). Introducing DNA into yeast by transformation. Methods; a companion to Methods in Enzymology, eds. M. Johnston and Stan Fields. Academic Press, Inc. 5:79-85

Schmidt, M., Geβner, G., Luff, M., Heiland, I., Wagner, V., Kaminski, M., Geimer, S., Eitzinger, N., Reiβenweber, T., Voytsekh, O., Fiedler, M., Mittag, M., Kreimer, G. (2006) Proteomic analysis of the eyespot of Chlamydomonas reinhardtii provides novel insights into its components and tactic movements. Plant Cell 18: 1908-1930.

Schroda, M., Blöcker, D., Beck, C. F. (2000) The HSP70A promoter as a tool for the improved expression of transgenes in Chlamydomonas. Plant J 21: 121-131.

Schroda, M., Vallon, O., Whitelegge, J. P., Beck, C. F., Wollman, F.-A. (2001) The chloroplastic GrpE homolog of Chlamydomonas: two isoforms generated by differential splicing. Plant Cell 13: 2823-2839.

Shao, N., Bock, R. (2008) A codon-optimized luciferase from Gaussia princeps facilitates the in vivo monitoring of gene expression in the model alga Chlamydomonas reinhardtii. Curr Genet 53: in press.

Shyu, Y. J., Liu, H., Deng, X., Hu, C.-D. (2006) Identification of new fluorescent protein fragments for bimolecular fluorescence complementation analysis under physiological conditions. Biotechniques 40: 61-66.

Sizova, I., Fuhrmann, M., Hegemann, P. (2001) A Streptomyces rimosus aphVIII gene coding for a new type phosphotransferase provides stable antibiotic resistance to Chlamydomonas reinhardtii. Gene 277(1-2): 221-229.

Southern, E. M. (1975). Detection of specific sequences among DNA fragments separated by gel electrophoresis, J Mol Biol. 98:503-517.

Suzuki, H., Saito, R. and Tomita, M. (2004). The ‘weighted sum of relative entropy’: a new index for synonymous codon usage bias. Gene 335:19-23.

Tsien, R. Y. 1998. The green fluorescent protein. Annu Rev Biochem. 67:509-44.

Walker, T. L., Purton, S., Becker, D. K., Collet, C. (2005) Microalgae as bioreactors. Plant Cell Rep 24: 629-641.

Weigel. D. and Glazebrook, J. (2006). Transformation of Agrobacterium Using Electroporation; CSH Protocols.

Wu-Scharf, D., Jeong, B., Zhang, C., Cerutti, H. (2000) Transgene and transposon silencing in Chlamydomonas reinhardtii by a DEAH-box (SEQ ID NO: 12) RNA helicase. Science 290: 1159-1162.

Zhang, J., Campbell, R. E., Ting, A. Y. and Tsien, R. Y. (2002). Creating new fluorescent probes for cell biology. Nat Rev Mol Cell Biol. 3:906-18.

Zimmer, M. (2002). Green fluorescent protein (GFP): applications, structure, and related photophysical behavior. Chem Rev. 102:759-81.

Zhang S, Raina S, Li H, Li J, Dec E, Ma H, Huang H, Fedoroff N V (2003) Resources for targeted insertional and deletional mutagenesis in Arabidopsis. Plant Mol Biol 53: 133-150.

1. A method of generating eukaryotic cells suitable for the expression of transgenes in said cells comprising:
(a) introducing a nucleic acid encoding a selectable marker responsive to a selecting agent into the nucleus of cells, wherein the level of expression of said selectable marker is proportional to the level of phenotypic responsiveness to said selecting agent;
(b) selecting, among the cells obtained in step (a), for cells with a detectable expression of said selectable marker;
(c) optionally propagating the cells selected for in step (b);
(d) mutagenizing the cells selected for in step (b) or propagated in step (c) or allowing for the appearance of spontaneous mutations in the cells selected for in step (b) or propagated in step (c);
(e) selecting for cells displaying an increased expression of said selectable marker compared to the expression obtained in step (b).

2. The method of claim 1, wherein the cell are plant cells, eukaryotic algal cells, fungal cells, yeast cells, or mammalian cells.

3. The method of claim 1, wherein the cells are Chlamydomonas cells.

4. The method of claim 1, wherein the responsiveness is resistance and wherein the selectable marker confers a resistance.

5. The method of claim 4, wherein the resistance gene is the CRY1-1 gene.

6. The method of claim 1, further comprising:
(a)′ introducing a nucleic acid encoding a selectable marker responsive to a selecting agent different than that applied in step (a) into the cells prior to step (b) and
(b)′ selecting for responsiveness to said selectable marker with said selecting agent preferably after step (a) and prior to step (b).

7. The method of claim 6, wherein the cells are auxotrophic for a compound, wherein the selectable marker is an auxotrophy gene encoding a protein restoring prototrophy for said compound and wherein step (b)′ comprises selecting for the restoration of prototrophy for said compound after step (a) and prior to step (b).

8. The method of claim 7, wherein the cells are Chlamydomonas cells which are auxotrophic for the Arg7 gene.

9. The method of claim 1, wherein mutagenesis is carried out by irradiation, chemical mutagenesis or genetic mutagenesis.

10. The method of claim 1, further comprising inactivating the selectable marker introduced in step (a) and optionally that in step (a)′ after step (e).

11. The method of claim 1, further comprising:
(f) introducing a nucleic acid molecule encoding a transgene of interest and optionally a selectable marker responsive to a selecting agent into the cells obtained in step (e); and
(g) assaying for expression of said transgene or a compound modulated by the expression product of said transgene in said cell, optionally in the presence of said selecting agent.

12. The method of claim 11, further comprising assaying for the presence of the complete transcription unit of the nucleic acid molecule encoding said transgene in the nucleus of the cell obtained in step (f) after introduction of said transgene.

13. A method of producing a compound of interest in a cell produced with the method of claim 1 comprising:
(a) introducing a nucleic acid encoding
(i) the compound of interest which is a protein or an RNA; or
(ii) a protein necessary to synthesize said compound of interest and optionally a selectable marker responsive to a selecting agent into said cell;
(b) expressing said protein in the cell; and
(c) isolating the compound of interest produced.

14. The method of claim 13, wherein the compound of interest is a pharmaceutical, a biofuel component, a diagnostic compound or a chemical.

15. A eukaryotic cell produced by the method of claim 1.

16. A kit comprising
a cell obtainable by the method of claim 1 and optionally a vector optimized for protein expression in said cell.

17. A method of detecting the expression and/or localization of a protein in the cell generated with the method of claim 1, comprising:
(a) expressing a nucleic acid encoding said protein fused to a reporter in said cell; or
(a)′ expressing a nucleic acid encoding said protein which is a reporter in said cell; and
(b) detecting the expression and/or localization of said reporter in said cell.

18. An in vitro method for detecting protein-protein interactions in the cell generated with the method of claim 1 comprising:
(a) expressing in said cell
i. a first nucleic acid encoding a fusion protein comprising a (poly)peptide of interest fused to a detectable marker and
ii. a second nucleic acid encoding a fusion protein comprising a (poly)peptide suspected of interacting with said first (poly)peptide fused to a different detectable marker; and
(b) detecting the localization of both detectable markers,
wherein a co-localization of both detectable markers in the cell is indicative of an interaction.

19. A kit comprising the cell of claim 15.


SEQUENCE LISTING (12 sequences)

SEQ ID NO: 1 - DNA, 19 bp, Artificial Sequence
  Description of Artificial Sequence Synthetic primer
catatggccaagggcgagg

SEQ ID NO: 2 - DNA, 25 bp, Artificial Sequence
  Description of Artificial Sequence Synthetic primer
gaattcttacttgtacagctcgtcc

SEQ ID NO: 3 - DNA, 61 bp, Artificial Sequence
  Description of Artificial Sequence Synthetic primer
gtgcattctaggaccccactgctactcacaacaagccccatggtgagcaagggcgaggagc

SEQ ID NO: 4 - DNA, 29 bp, Artificial Sequence
  Description of Artificial Sequence Synthetic primer
gaattcttacttgatcagctcgtccatgc

SEQ ID NO: 5 - DNA, 25 bp, Artificial Sequence
  Description of Artificial Sequence Synthetic primer
aaggatccccgggcgcgccagaagg

SEQ ID NO: 6 - DNA, 25 bp, Artificial Sequence
  Description of Artificial Sequence Synthetic primer
ggcggccatatgaagatgttgagtg

SEQ ID NO: 7 - DNA, 25 bp, Artificial Sequence
  Description of Artificial Sequence Synthetic primer
cctcagaagaactcgtccaacagcc

SEQ ID NO: 8 - DNA, 25 bp, Artificial Sequence
  Description of Artificial Sequence Synthetic primer
ggaggatctggacgaggagcggaag

SEQ ID NO: 9 - DNA, 24 bp, Artificial Sequence
  Description of Artificial Sequence Synthetic primer
cgagcccttcgaacagccaggccg

SEQ ID NO: 10 - DNA, 17 bp, Artificial Sequence
  Description of Artificial Sequence Synthetic primer
gtaaaacgacggccagt

SEQ ID NO: 11 - protein, 6 aa, Artificial Sequence
  Description of Artificial Sequence Synthetic 6xHis tag
HHHHHH

SEQ ID NO: 12 - protein, 4 aa, Artificial Sequence
  Description of Artificial Sequence Synthetic peptide
DEAH